(19) United States
(12) Patent Application Publication
(10) Pub. No.: US 2008/0187937 A1
(43) Pub. Date: Aug. 7, 2008

(54) Method For Measuring Dynamics Of Self-Assembling Systems Of Biological Molecules In Vivo And Uses For Discovering Or Evaluating Therapeutic Agents

(76) Inventors: Marc K. Hellerstein, Kensington, CA (US); Patrizia Fanara, Oakland, CA (US)

Correspondence Address: MORGAN, LEWIS & BOCKIUS, LLP, ONE MARKET SPEAR STREET TOWER, SAN FRANCISCO, CA, 94105, US

(21) Appl. No.: 11/573,374

(22) PCT Filed: Aug. 8, 2005
(86) PCT No.: PCT/US05/28069

§ 371 Date: Oct. 17, 2007

Related U.S. Application Data
(60) Provisional application No. 60/599,716, filed on Aug. 7, 2004.

(51) Int. Cl. G01N 33/567; G01N 33/53

(52) U.S. Cl. 435/7.2

(57) ABSTRACT

The Applicants have established a simple, rapid assay of measuring the dynamics of self-assembling systems of biological molecules, based on stable isotope labeling technology that can be used in intact animals including humans. Examples of self-assembling systems of biological molecules include microtubule polymers, actin filaments, amyloid-beta plaques or fibrils, prion plaques or fibrils, fibrin aggregates, tau filaments (e.g., neurofibrillary tangles), α-synuclein filaments, and mutant hemoglo-bin aggregates. The method reveals constitutive differences in the dynamics of assembly and disassembly between tissues and is sensitive to the action of compounds that stabilize these dynamics.

### CROSS-REFERENCE TO RELATED APPLICATIONS

This application claims priority to U.S. provisional application No. 60/599,716 filed on Aug. 7, 2004, which is hereby incorporated by reference in its entirety.

### FIELD OF THE INVENTION

The invention relates to methods for measuring the dynamics or rates of assembly and disassembly of self-assembling systems of biological molecules (e.g., “molecular assemblages”). Such systems include, inter alia: microtubule polymers from tubulin dimers; amyloid-β peptides or plaques in the brain from amyloid-β proteins; sickle cell hemoglobin aggregates in erythrocytes from mutant hemoglobin proteins; prion fibrils or plaques in the brain from prion proteins; fibrin assemblages (e.g., blood clots) from fibrin proteins; actin filaments from actin proteins; tau filaments (neurofibrillary tangles) from tau proteins; α-synuclein filaments or aggregates (Lewy bodies and Lewy neurites) from α-synuclein proteins; cardiolipin aggregates in the mitochondrial inner membrane from cardiolipin molecules; cholesterol aggregates in the plasma membrane from cholesterol molecules; phospholipid aggregates in the mitochondrial inner membrane from phospholipid molecules; phospholipid aggregates in the nuclear membrane from phospholipid molecules; phospholipid aggregates in the plasma membrane from phospholipid molecules; phospholipid aggregates in the myelin lamella from phospholipid molecules; and galactocerebroside aggregates in the myelin lamella from galactocerebroside molecules. The methods are applicable in drug discovery and development and in identifying drug toxicity.

### BACKGROUND OF THE INVENTION

There are a variety of self-assembling systems of biological molecules in cells or organisms (herein termed “molecular assemblages”). These molecular assemblages, although diverse in structure and content, have a common feature in that they are formed without the need for catalysts such as enzymes, e.g., they “self assemble.” An important distinction to recognize in this context is the difference between a self-assembling system of biological molecules and most other macromolecules, which are formed via biosynthetic processes in living organisms. Self-assembling systems differ in several ways from standard biosynthetic formation of other types of macromolecules, such as polynucleotides (DNA, RNA), proteins, lipids, complex carbohydrates, etc. Standard macromolecules that are not of the self-assembling type require an enzyme or catalyst or an entire molecular machinery to be synthesized (e.g., a ribosomal apparatus for protein synthesis; a complex, multi-enzyme apparatus for DNA replication or transcription; an endoplasmic reticulum for lipid biosynthesis,etc.); have an energy barrier that prevents spontaneous formation in vivo; and are characterized typically by covalent chemical bonds between the subunits that form into the macromolecule; and do not disassemble spontaneously but require a separate catabolic apparatus (e.g., proteosomes and proteolytic enzymes for disassembly of proteins; oxidative enzymes and a mitochondrial matrix for catabolic disassembly of long-chain fatty acids; RNAses for RNA disassembly; etc.). Self-assembling systems of biological molecules or molecular aggregates, in contrast, do not require enzymes, catalysts or a molecular machinery to form, do not typically have an energy barrier to formation but do so spontaneously in vivo, need not have covalent chemical bonds between subunits in the assemblage, and do disassemble spontaneously in vivo. The biological roles, functions and significance of self-assembling systems of biological molecules thereby differ radically from the roles, functions and significance of macromolecules that are not of the self-assembling type. One example that can be used to illustrate the concept of a self-assembling molecular assemblage is the microtubule. Microtubules are composed of dimeric subunits, which in turn contain a α-tubulin and a β-tubulin monomeric protein. Once formed, the αβ-dimers are very stable. These dimeric subunits self-assemble into long chains, or filaments, termed microtubules, which then function as a microskeleton in living cells.

Microtubules exhibit polarity in their structure with a plus end (fast growing) and a minus end (slow growing). Microtubules may vary in their rates of assembly and disassembly, e.g., they are dynamic (“microtubule dynamics”). Microtubules in cells are believed to exist in a state of continued flux (assembly/disassembly, also known as polymerization/depolymerization). The balance of assembly versus disassembly within this context of microtubule dynamics thereby determines the mean length of microtubules and their lifespan in the cell.

There are a variety of other molecular assemblages that exist within or outside the cell that can be characterized by dynamic assembly and disassembly. Examples include mutant hemoglobin aggregates, amyloid-beta (Aβ) fibrils or plaques, fibrin assemblages comprising blood clots, prion fibrils or plaques, α-synuclein filaments or aggregates (Lewy bodies or Lewy neurites, tau filaments or aggregates (neurofilamentary tangles), phospholipid aggregates, cardiolipin aggregates, and cholesterol aggregates. Until now, there has not been a rapid, precise, and accurate method for measuring the dynamic assembly and disassembly of molecular assemblages. Such a method would find use in drug discovery and development, diagnosis and prognosis of a variety of diseases associated with molecular assemblages, and basic biomedical research.

### SUMMARY OF THE INVENTION

The present invention provides methods for measuring the dynamics (i.e., the rates of assembly and disassembly) of molecular assemblages. The method is applicable to such self-assembling systems of biological molecules as actin polymerization into filamentous actin (e.g., microfilaments), amyloid beta (Aβ) aggregation into Aβ fibrils or plaques, prion aggregation into prion fibrils or plaques, fibrin aggregation into fibrin assemblages (e.g., blood clots), mutant hemoglobin aggregation into hemoglobin aggregates in sickled erythrocytes, tau aggregation into tau filaments (neurofilamentary tangles), α-synuclein aggregation into α-synuclein filaments and aggregates (Lewy bodies and Lewy neurites), cardiolipin aggregates in the inner membrane of the mitochondria, cholesterol aggregates in the plasma membrane, galactocerebroside aggregates in the myelin lamella, and phospholipid aggregates in the plasma membrane, myelin lamellae, and the membranes of subcellular organelles.

The present invention demonstrates, for the first time, microtubule dynamics and the effect of microtubule-targeted tubulin-polymerizing agents (MTPAs) in an in vivo setting.

The method is amenable to high-throughput screening of compounds for measuring, identifying, evaluating, and characterizing effects on microtubule dynamics and the dynamics of other self-assembling systems of biological molecules, and thus for identifying new therapeutic agents such as anticancer agents, and/or evaluating toxicity and other effects.

The invention allows for the evaluation and/or quantitation of the dynamics of assembly and disassembly of microtubules and/or other self-assembling systems of biological molecules measured from living systems. As outlined herein, the evaluation can be done in a variety of ways. In one aspect, the invention provides methods of comparison of dynamics between living systems that have been exposed to one or more candidate agents to the dynamics of microtubules and other self-assembling systems of biological molecules measured from non-exposed living systems. Non-exposed living systems may be living systems having a disease such as cancer or another proliferative disorder (such as psoriasis) but not yet having been exposed to the candidate agent(s), or non-exposed living systems may be living systems not having cancer or another proliferative disorder. Non-proliferative disorders can be evaluated in the same way. Differences between the dynamics of microtubules or the dynamics of other self-assembling systems of biological molecules from the exposed and non-exposed living systems are identified and this information is then used to determine whether the one or more candidate agents (or combinations or mixtures thereof elicit a change in microtubule dynamics or a change in dynamics of other self-assembling systems of biological molecules in the exposed living system. The one or more compounds may be administered to a mammal and the microtubule dynamics (rates) or the dynamics (rates) of other self-assembling systems of biological molecules may be calculated and evaluated against the dynamics (rates) calculated from an unexposed mammal of the same species. Alternatively, the microtubule dynamics (rates) or the dynamics (rates) of other self-assembling systems of biological molecules from the same mammal may be calculated prior to exposure of the one or more compounds and then the dynamics (rates) may be calculated in the same mammal after exposure to said one or more compounds and then compared. The mammal may be a human.

In one aspect, the invention provides methods for measuring the rate of self assembly of subunits into biological molecular assemblages in a test living system as compared to a control living system. The method comprises administering an isotope-labeled substrate to the living system for a first period of time sufficient for the substrate to be incorporated into at least one of the subunits and at least one of the molecular assemblages. A sample is obtained from the living system, and the amount of labeled molecular assemblages from the first sample is quantified. Optionally, particularly in the case of microtubule evaluation, the amount of unincorporated labeled subunits is quantified as well. The amount of labeled molecular assemblages is compared to the amount of labeled molecular assemblage in a control living system, and optionally, the amount of unincorporated labeled subunits is compared to the amount of unincorporated labeled subunits in a control living system, to determine a difference in the rate of self assembly in the test living system as compared to the control living system.

In an additional aspect, the comparing step comprises calculating the molecular flux rate of the labeled molecular assemblages and the free subunits, such that the comparing step calculates the ratio of the rates and compares that ratio to the ratio of molecular flux rates in said control living system.

In a further aspect, the methods further comprise administering a candidate agent to the test living system, either prior, during or after the administration of the isotope-labeled substrate(s).

Additional aspects of the invention include administering the substrate for a second period of time and repeating the calculations; obtaining a second sample and repeating the calculations; administering a second substrate; or combinations thereof.

In one embodiment, dynamics of self-assembling systems of biological molecules are measured by use of stable isotope labeling techniques. Said techniques involve the administration or contacting of stable isotope-labeled substrates to a biological system of interest. The stable isotope label may include ²H, ¹³C, ¹⁵N, ¹⁸O, ³³S, ³⁴S. In another embodiment, the microtubule dynamics (rates) or the dynamics (rates) of other self-assembling systems of biological molecules are measured by use of radioactive isotope labeling techniques. The radioactive isotope may include ³H, ¹⁴C ³²P, ³³P, ³⁵S, ¹²⁵I, ¹³¹I.

Isotope labeled substrates include, but are not limited to ²H₂O, H₂¹⁸O, ¹⁵NH₃, ¹³CO₂, H¹³CO₃, ²H-labeled amino acids, ¹³C-labeled amino acids, ¹⁵N-labeled amino acids, ¹⁸O-labeled amino acids, ³⁴S or ³³S-labeled amino acids, ³H₂O, ³H-labeled amino acids, and ¹⁴C-labeled amino acids, ²H-glucose, ¹³C-labeled glucose, ²H-labeled organic molecules, ¹³C-labeled organic molecules, and ¹⁵N-labeled organic molecules.

In one embodiment, the incorporation of stable isotope-labeled substrates into one or more tubulin dimers (subunits of microtubule polymers) and the incorporation into microtubule polymers are measured concurrently by methods known in the art.

In another embodiment of the invention, isotopically perturbed molecules are provided, said isotopically perturbed molecules comprising one or more stable isotopes. The isotopically perturbed molecules are products of the labeling methods described herein.

In yet another embodiment of the invention, the isotopically perturbed molecules are labeled with one or more radioactive isotopes.

In yet another embodiment of the invention, one or more kits are provided that comprise isotope-labeled precursors and instructions for using them. The kits may contain stable-isotope labeled precursors or radioactive-labeled isotope precursors or both. Stable-isotope labeled precursors and radioactive-labeled isotope precursors may be provided in one kit or they may be separated and provided in two or more kits. The kits may further comprise one or more tools for administering the isotope-labeled precursors. The kits may also comprise one or more tools for collecting samples from a subject.

In yet another embodiment of the invention, one or more information storage devices are provided that comprise data generated from the methods of the present invention. The data may be analyzed, partially analyzed, or unanalyzed. The data may be imprinted onto paper, plastic, magnetic, optical, or other medium for storage and display.

In yet another embodiment of the invention, one or more drug agents identified and at least partially characterized by the methods of the present invention are contemplated.

### BRIEF DESCRIPTION OF THE DRAWINGS

FIGS. 1A and 1B depict pathways of labeled hydrogen (²H or ³H) exchange from isotope-labeled water into selected free amino acids. Two NEAA's (alanine, glycine) and an EAA (leucine) are shown, by way of example. Alanine and glycine are presented in FIG. 1A. Leucine is presented in FIG. 1B. Abbreviations: TA, transaminase; PEP-CK, phosphoenolpyruvate carboxykinase; TCAC, tricarboxylic acid cycle; STHM, serine tetrahydrofolate methyltransferase. FIG. 1C depicts ¹⁸O-labeling of free amino acids by H₂¹⁸O for protein synthesis.

FIG. 2A shows the incorporation of ²H-labeled tubulin dimers into microtubule polymers.

FIG. 2B shows the effects of microtubule-targeted tubulin-polymerizing agents on this process.

FIG. 3A shows microtubule dynamics in actively proliferating human lung cancer cells (SW1573). Cells were cultured and labeled for 36 hours in media containing 4% ²H₂O. ²H label steadily increased during the 36 hour time period reflecting ²H-alanine incorporation into newly synthesized tubulin dimer and polymer franctions. FIG. 3B shows microtubule dynamics in the same cell line (SW1573), which was treated with 0.4 mM paclitaxel. The presence of paclitaxel reduced ²H-enrichment in labeled polymers reflecting inhibition of ²H-tubulin dimer incorporation into polymer.

FIG. 4A shows a dose-response experiment with paclitaxel in nude mice implanted with SW1573 human lung cancer cells. Mice were injected i.p. with the indicated amounts of paclitaxel and labeled with ²H₂O for 24 hours. At ≧5 mg/kg, paclitaxel increased de novo synthesis of tubulin dimer but inhibited label incorporation into polymer. FIG. 4B shows a dose-response experiment with paclitaxel in nude mice implanted with MCF-7 human breast cancer cells. Mice were injected i.p. with the indicated amounts of paclitaxel and labeled with ²H₂O for 24 hours. Like the response seen in SW1537 cells (FIG. 4A) at ≧5 mg/kg, paclitaxel increased de novo synthesis of tubulin dimer but inhibited label incorporation into polymer.

FIG. 5A shows the results from nude mice implanted with human SW1573 lung cancer cells, injected with varying doses of paclitaxel, and labeled with ²H₂O for 24 hours. Microtubule dynamics were expressed as (²H enrichment in polymer)/(²H enrichment in dimer). Inhibition of de novo DNA synthesis was quantified as the reduction in ²H label incorporation into tumor cell DNA. As can be seen, there is a strong correlation between inhibition of microtubule dynamic instability and reduction in cell proliferation. FIG. 5B shows the results from nude mice implanted with human MCF-7 breast cancer cells, injected with varying doses of paclitaxel, and labeled with ²H₂O for 24 hours. Microtubule dynamics were expressed as (²H enrichment in polymer)/(²H enrichment in dimer). Inhibition of de novo DNA synthesis was quantified as the reduction in ²H label incorporation into tumor cell DNA. Like the SW1573 data (FIG. 5A), there is a strong correlation between inhibition of microtubule dynamics and reduction in cell proliferation.

FIG. 6 shows a dose-response effect of paclitaxel on microtubule dynamics in the peripheral nervous system. Sciatic nerve microtubule dynamics are shown in 6A and densitometric quantification of tubulin dimer and microtubule polymer levels are depicted in 6B.

FIG. 7 illustrates uses of the invention herein in a drug discovery and development process.

### DETAILED DESCRIPTION OF THE INVENTION

### Overview of the Invention

The Applicants have established a simple, rapid assay for measuring the dynamics of self-assembling systems of biological molecules (i.e., the rates of assembly and disassembly of molecular assemblages), based on isotope labeling technology (including both radioisotopes and stable isotopes) that can be used in living systems, including cells (e.g., single cells or in vitro cellular systems) and organisms (e.g. intact animals and humans). That is, the invention utilizes the natural equilibrium of “monomers” into self-assembling “polymers”; thus, the invention allows for the comparison between the dynamics of assembly and disassembly. Such self-assembling systems of biological molecules (e.g., “molecular assemblages”) include: microtubules (formed from the polymerization of tubulin dimers); filamentous actin or microfilaments (formed from the polymerization of monomeric actin); amyloid-beta (Aβ) fibrils or plaques (formed from the aggregation of Aβ proteins); tau filaments or plaques (neurofibrillary tangles) formed from the aggregation of tau proteins; α-synuclein filaments or aggregates (Lewy bodies or Lewy neurites) formed from the aggregation of α-synuclein proteins; mutant hemoglobin aggregates (formed from the aggregation of mutant hemoglobin in sickled erythrocytes); prion fibrils or plaques (formed from the aggregation of mutant or infectious prion proteins); fibrin aggregates or blood clots (formed from the polymerization of fibrin); galactocerebroside aggregates in the myelin lamella; diphosphatidylglycerol (e.g., cardiolipin) aggregates in the inner membrane of mitochondria; cholesterol aggregates in the plasma membrane; and phospholipid aggregates (e.g., aggregates of phosphoglycerides including aggregates of phosphatidylserine, phosphatidylcholine, phosphatidylinositol, and phosphatidylethanolamine) in the plasma membrane, myelin lamellae, and in membranes of subcellular organelles.

An important distinction to recognize in this context is the difference between a self-assembling system of biological molecules and most other macromolecules, which are formed via biosynthetic processes in living organisms. Self-assembling systems differ in several ways from standard biosynthetic formation of other types of macromolecules, such as polynucleotides (DNA, RNA), proteins, lipids, complex carbohydrates, etc. Standard macromolecules that are not of the self-assembling type require an enzyme or catalyst or an entire molecular machinery to be synthesized (e.g., a ribosomal apparatus for protein synthesis; a complex, multi-enzyme apparatus for DNA replication or transcription; an endoplasmic reticulum for lipid biosynthesis,etc.); have an energy barrier that prevents spontaneous formation in vivo; and are characterized typically by covalent chemical bonds between the subunits that form into the macromolecule; and do not disassemble spontaneously but require a separate catabolic apparatus (e.g., proteosomes and proteolytic enzymes for disassembly of proteins; oxidative enzymes and a mitochondrial matrix for catabolic disassembly of long-chain fatty acids; RNAses for RNA disassembly; etc.). Self-assembling systems of biological molecules or molecular aggregates, in contrast, do not require enzymes, catalysts or a molecular machinery to form, do not typically have an energy barrier to formation but do so spontaneously in vivo, need not have covalent chemical bonds between subunits in the assemblage, and do disassemble spontaneously in vivo. The biological roles, functions and significance of self-assembling systems of biological molecules thereby differ radically from the roles, functions and significance of macromolecules that are not of the self-assembling type.

In general, the invention can be practiced in a variety of different ways. In one embodiment, as is more fully outlined below, the administration of an isotope-labeled substrate into a living system (for example an individual with a disease, an individual that has been exposed to a drug or candidate drug, a cell line of interest, primary cancer cells, bacteria, etc.) results in the incorporation of the label into the molecular assemblages, e.g. cytoskeletal or flagellular microtubules. Measurements taken in one experiment on one system can be compared to control data, e.g. control living systems without the disease or drug. Measurements can be taken in a variety of ways, as is more fully outlined below. Thus, for example, the ratio of incorporated substrate:unincorporated substrates at one or more time points can be compared to control systems. Differential incorporation (and thus different ratios) are an indication of the modulation of the quantity or rate of self-assembly activity. This can be done as a sampling of the system at a single time point after administration of the isotope-labeled substrate (either as a continuous or discontinuous administration), or with multiple sampling at a plurality (e.g. two or more) time points. In addition, a single sampling can be done after a single administration, or single sampling can be done after administration for different times. For example, administration of a short bolus of label, followed by incubation and a sampling at time X can be done; the system is then allowed to clear, and a second administration of a longer bolus can be done, followed again by incubation and sampling. Alternatively, multiple samplings and multiple administrations (including the use of multiple isotope labels and/or different substrates) can be done.

Alternatively, the amount of label incorporation can be used to calculate the molecular flux rate for the increase of the labeled substrates into assemblages. This can also be done as outlined above using single sampling at a single time point (and calculating the rate based on the zero time point) or with multiple sampling, multiple time points or both. Ratios of molecular flux rates are then compared to elucidate alterations in self-assembly activity.

The assay is readily adaptable for human use, as labeling with ²H₂O or other stable isotope labels can be performed safely and easily in humans. In addition, the assay of modulation of self-assembly of molecular assemblages can be combined with other assays, for example of new nucleic acid synthesis, appearance of apoptosis, angiogenesis, etc., to allow the elucidation of mechanistic pathways and combination therapies, among a variety of other utilities as outlined herein.

When applied to microtubule dynamics, the methods of the present invention reveal constitutive differences in the rates of assembly and disassembly (polymerization and depolymerization) of microtubules between tissues, and is exquisitely sensitive to the action of microtubule-stabilizing agents, having been demonstrated in practice to reveal significant effects at doses up to 30-fold lower than the maximum tolerated therapeutic dose of paclitaxel. When used in combination with another assay, such as the simultaneous, stable isotope-based measurements of new DNA synthesis, the methods of the present invention can reveal quantitatively mechanism-based therapeutic and toxic actions of microtubule-stabilizing drugs, both in actively proliferating and post-mitotic tissues, and provide information about variation among tumors with regard to susceptibility to downstream effects of such drugs. These features have important implications for the mechanism of disruptive action of anti-mitotic drugs and opens new frontiers for drug discovery and development. It should be noted that in some instances, therapeutic effects are seen with assemblage stability (e.g. lack of new growth or polymerization), assemblage disassembly (e.g. blood clots and arterialschlerotic plaques), or assemblage assembly.

The methods disclosed herein are applicable to self-assembling systems of biological molecules (e.g., molecular assemblages). As indicated supra, such self-assembling systems of biological molecules in biology and pathology include, microtubule polymers, including cytoskeletal microtubules as well as cilular and flagellular microtubules, the self-assembly and disassembly (polymerization and depolymerization) of actin filaments (microfilaments), the primary constituent of the cytoskeleton, defects or dysregulation of which are associated with cardiomyopathies, muscular dystrophies, ischemic acute renal failure, cancer and angiogenesis, myasthenia gravis, and amyotrophic lateral sclerosis, among others; the self-assembly and disassembly of A□ fibrils or plaques from A□ peptides, a process that occurs in the brains of Alzheimer's patients and animal models of Alzheimer's disease; the self-assembly and disassembly of tau filaments or plaques (e.g., neurofibrillary tangles), a process which occurs in the brains of Alzheimer's patients and in patients with dementia, and in animal models; the self-assembly and disassembly of □-synuclein filaments or aggregates (Lewy bodies or Lewy neurites), a process which occurs in the brains of Parkinson's disease patients and in the brains of dementia patients, and in animal models; the self-assembly and disassembly of mutant hemoglobin aggregates from free hemoglobin in erythrocytes of patients with sickle cell disease, a process that leads to end-organ dysfunction in this condition; the self-assembly and disassembly of fibrin aggregates (blood clots) from free fibrin, defects in this process leading to blood clotting disorders; the self-assembly and disassembly of prion fibrils or plaques from free PrP in the brain in Creutzfeldt-Jakob disease, kuru, scrapies, or bovine spongiform encephalopathy; the self-assembly and disassembly of cardiolipin aggregates in the inner membranes of mitochondria; the self-assembly and disassembly of cholesterol aggregates in the plasma membrane; the self-assembly and disassembly of galactocerebroside aggregates in the myelin lamella, which occurs in patients with multiple sclerosis and other demyelinating diseases, and in animal models; and the self-assembly and disassembly of phospholipid aggregates (e.g., aggregates of phosphoglycerides including aggregates of phosphatidylserine, phosphatidylcholine, phosphatidylinositol, and phosphatidylethanolamine) in the plasma membrane, myelin lamellae, and in the membranes of subcellular organelles. These systems contrast to or are distinguished from systems of biological molecules that are not self assembling but require catalysts or other exogenous factors to form higher level structures, e.g., deoxyribonucleotides, amino acids, etc.

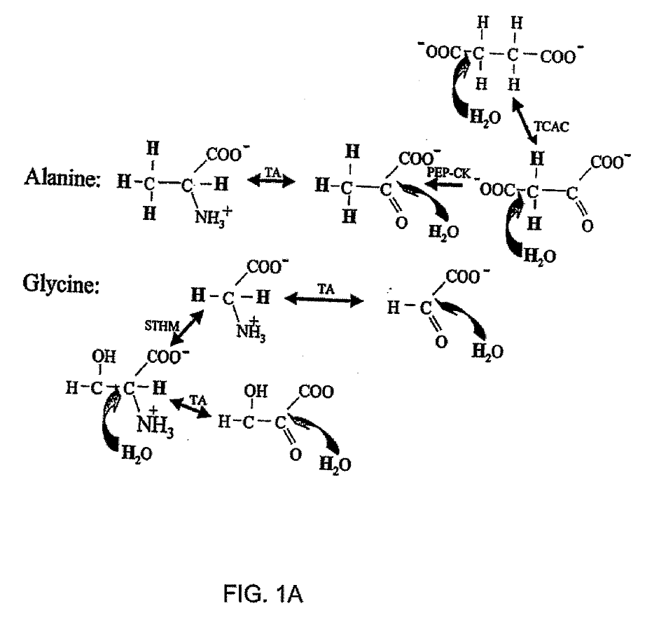

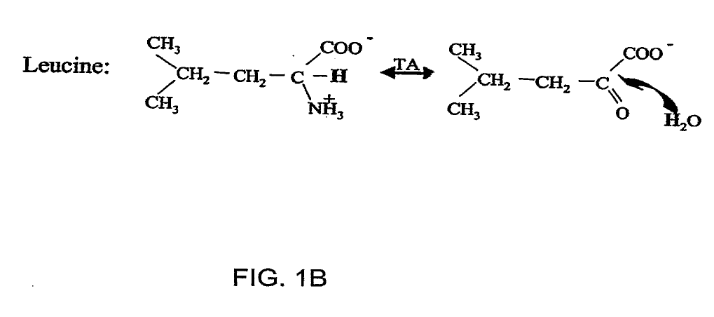

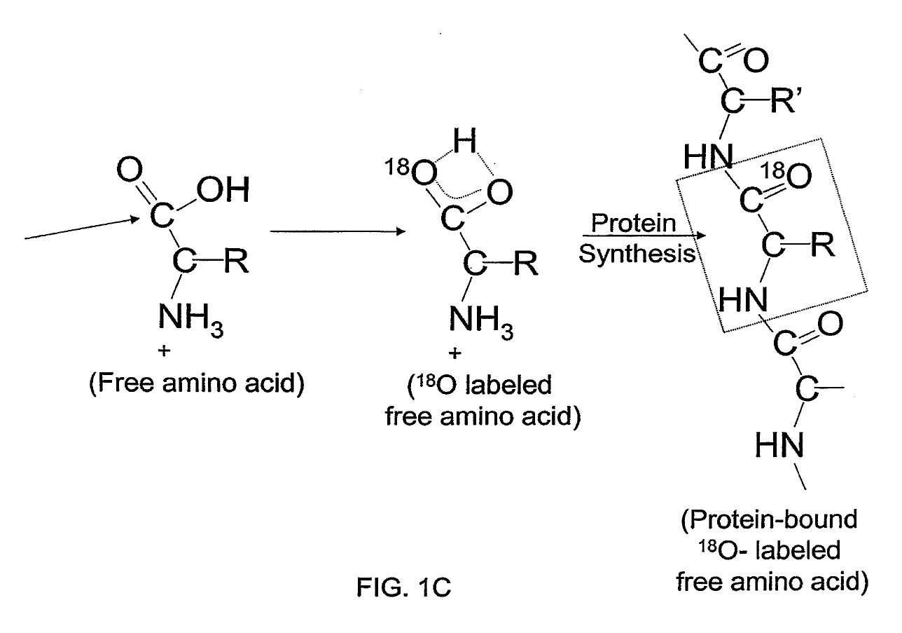

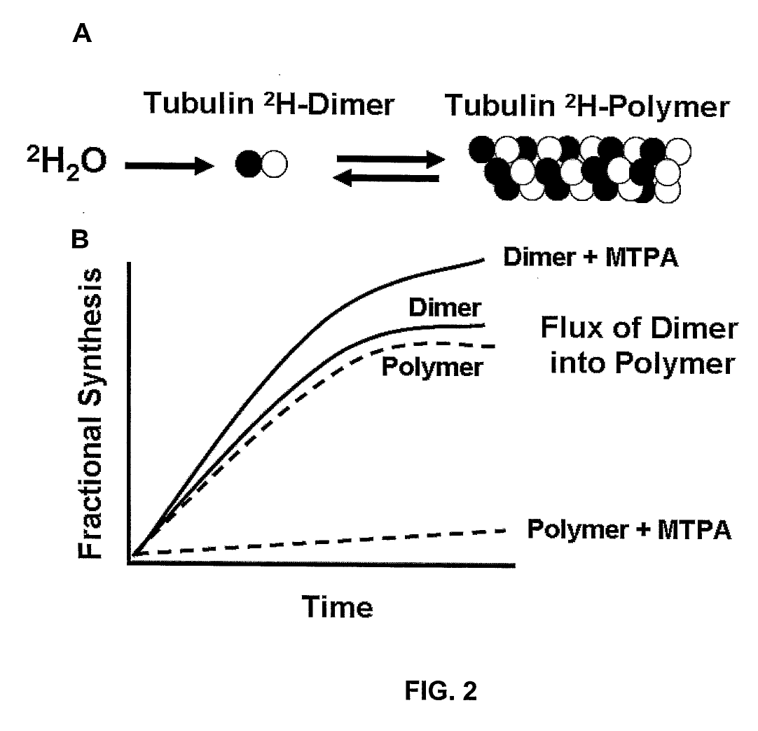

In one embodiment, the methods of the present invention makes use of deuterated water (²H₂O) to isotopically label free tubulin subunits (see FIGS. 1A, 1B) or other free subunits of self-assembling systems of biological molecules. The skilled artisan will appreciate that other isotopes may be used and may be administered via labeled amino acids or labeled fatty acids or labeled glycerol or other precursors of protein biosynthesis and phospholipid biosynthesis as described more fully, infra (also see FIG. 1C). Staying with the ²H₂O example and the application to microtubules, the ²H label enters newly synthesized free tubulin subunits via metabolic pathways for the biosynthesis of nonessential amino acids. Incorporation of ²H into newly synthesized free tubulin subunits appears in the tubulin dimer pool before being incorporated through polymerization into microtubules (see FIG. 2A). In biological settings in which microtubules are highly dynamic, ²H-label accumulates in the dimer and polymer pools at similar rates reflecting rapid exchange kinetics between the two pools (e.g., dynamic instability of the microtubules). However, in settings where microtubules are stabilized by microtubule-targeted tubulin-polymerizing agents (MTPAs) or by endogenous microtubule-stabilizing factors, ²H-label in newly synthesized tubulin appears in the dimer pool at a rate proportional to the biosynthetic rate of free tubulin, whereas incorporation into microtubule polymers is slower, often dramatically so (see FIGS. 2B-4).

Microtubule dynamics are then quantified by gas chromatography/mass spectrometry (GC/MS) or other analytical techniques known in the art (discussed more fully, infra). This methodology requires significantly less compound, is predictive in a matter of just days, and is highly reproducible.

As the skilled artisan will appreciate, the methods of the present invention can be applied to other self-assembling systems of biological molecules including those listed, supra and infra.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999). Furthermore, procedures employing commercially available assay kits and reagents will typically be used according to manufacturer-defined protocols unless otherwise noted.

The practice of the invention will additionally utilize, unless otherwise indicated, conventional techniques of chemistry and analytic chemistry which are within the skill of the art. Such techniques are fully explained in the literature, for example, in Fundamentals of Analytical Chemistry (D. Skoog, D West, F Holler, S Crouch, auth, 2003); Analytical Chemistry (S. Higson, auth, 2004); Advanced Instrumental Methods of Chemical Analysis (J. Churacek, ed, 1994); and Advanced mass spectrometry: Applications in organic and analytical chemistry (U. Schlunegger).

The practice of the invention will additionally utilize, unless otherwise indicated, conventional techniques of pre-clinical and clinical research which are within the skill of the art. Such techniques are fully explained in the literature.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

“Molecular flux rates” refers to the rate of synthesis and/or breakdown of a protein and/or organic metabolite. “Molecular flux rates” also refers to a protein and/or organic metabolite's input into or removal from a pool of molecules, and is therefore synonymous with the flow into and out of said pool of molecules.

“Metabolic pathway” refers to any linked series of two or more biochemical steps in a living system (e.g., a biochemical process), the net result of which is a chemical, spatial or physical transformation of a molecule or molecules. Metabolic pathways are defined by the direction and flow of molecules through the biochemical steps that comprise the pathway. Molecules within metabolic pathways can be of any biochemical class, e.g., including but not limited to lipids, proteins, amino acids, carbohydrates, nucleic acids, polynucleotides, porphyrins, glycosaminoglycans, glycolipids, intermediary metabolites, inorganic minerals, ions, etc.

“Dynamics” refers to the kinetic features of a molecule or system of molecules. As used herein, it refers to chemical rates in the dimension of time (e.g., mass or moles per unit of time, for example moles per minute, grams per hour) and includes synthesis rates, breakdown rates, turnover rates, transformation rates, interchange rates, assembly and disassembly rates, polymerization and depolymerization rates, aggregation and disaggregation rates, and other aspects of the kinetic behavior of molecular assemblages. It should be noted that some of these rates can be related; e.g. the rate of synthesis and the rate of breakdown can be combined to give the turnover rate, etc. This is also referred to in some instances as “the flux rate through a metabolic pathway”. In some cases, in particular for non-reversible or very slow depolymerizations, “flux rate through a pathway” can refer to the transformation rate from a clearly defined biochemical starting point (e.g. introduction of mutant hemoglobin) to a clearly defined biochemical endpoint (e.g. irreversible aggregation).

“Self-assembling system of biological molecules” or “molecular assemblages” refers to any biochemical or molecular entity that is comprised of an assemblage of subunits (e.g. “monomers”), and that is capable of forming said assemblages (“polymers”) spontaneously, and typically dissolving (degrading) said assemblage spontaneously, without a need for catalysts such as enzymes, complex molecular machinery, biochemical activation, or other external factors (except to the extent that correct pH, temperature, etc. is needed for the living system). In some cases, the polymerization or assembly occurs without the addition of energy, e.g. ATP. The rates of assembly and disassembly of said self-assembling systems of biological molecules may be modified by external factors (modulators, drugs, etc.), but these factors are not required for the self-assembly or disassembly to occur in vivo. Assemblages in the present invention are generally but not always homopolymeric, such as in the case of tubulin formation of microtubules, but heteropolymeric assemblages are also included. The assemblage may take the biochemical form of linear or branched polymers, fibers, fibrils, filaments, aggregates, or other higher-order structures of subunits. As used herein, the term “molecular assemblage” is synonymous with “self-assembling system of biological molecules.”

“Molecular assemblages” include, but are not limited to, microtubules (formed from the polymerization of tubulin dimers); filamentous actin or microfilaments (formed from the polymerization of monomeric actin); amyloid-beta (Aβ) fibrils or plaques (formed from the aggregation of Aβ proteins); tau filaments or plaques (neurofibrillary tangles) formed from the aggregation of tau proteins; α-synuclein filaments or aggregates (Lewy bodies or Lewy neurites) formed from the aggregation of α-synuclein proteins; mutant hemoglobin aggregates (formed from the aggregation of mutant hemoglobin, for example in sickled erythrocytes); prion fibrils or plaques (formed from the aggregation of mutant or infectious prion proteins); fibrin aggregates or blood clots (formed from the polymerization of fibrin); galactocerebroside aggregates in the myelin lamella; diphosphatidylglycerol (e.g., cardiolipin) aggregates in the inner membrane of mitochondria; cholesterol aggregates in the plasma membrane; and phospholipid aggregates (e.g., aggregates of phosphoglycerides including aggregates of phosphatidylserine, phosphatidylcholine, phosphatidylinositol, and phosphatidylethanolamine) in the plasma membrane, myelin lamellae, and in membranes of subcellular organelles.

As used herein, “subunit” or “subunit of self-assembling system of biological molecules” refers to the free (non-assembled) form of the system of biological molecules of interest, which is capable of self-assembling into polymers, fibers, filaments, fibrils, aggregates, or other higher-order structures of subunits (e.g., molecular assemblages). These are also referred to herein sometimes as “precursors” or “monomers”.

“Self-assembly and disassembly” refers to any self-formation (“self-assembly”) and any self-degradation (“disassembly”) of a self-assembled biological molecule in a self-assembling system. Such self-assembly and disassembly includes, but is not limited to, polymerization and depolymerization; and aggregation and disaggregation.

“Isotopes” refer to atoms with the same number of protons and hence of the same element but with different numbers of neutrons (e.g., ¹H vs. ²H or D). The term “isotope” includes “stable isotopes”, e.g. non-radioactive isotopes, as well as “radioactive isotopes”, e.g. those that decay over time, with the former being preferred in some embodiments. The stable isotope label may include, but are not limited to, ²H, ¹³C, ¹⁵N, ¹⁸O, ³³S, ³⁴S. The radioactive isotope may include, but is not limited to, ³H, ¹⁴C, ³²P, ³P, ³⁵S, ¹²⁵I, ¹³¹I.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition therefore each isotopologue has a unique exact mass but may not have a unique structure. An isotopologue usually includes of a family of isotopic isomers (isotopomers) which differ by the location of the isotopes on the molecule (e.g., CH₃NHD and CH₂DNH₂ are the same isotopologue but are different isotopomers).

“Isotope-labeled water” includes water labeled with one or more specific heavy isotopes of either hydrogen or oxygen. Specific examples of isotope-labeled water include ²H₂O, ³H₂O, and H₂¹⁸O.

Isotope labeled substrates include, but are not limited to ²H₂O, H₂¹⁸O, ¹⁵NH₃, ¹³CO₂, H¹³CO₃, ²H-labeled amino acids, ¹³C-labeled amino acids, ¹⁵N-labeled amino acids, ¹⁸O-labeled amino acids, ³⁴S or ³³S-labeled amino acids, ³H₂O, ³H-labeled amino acids, and ¹⁴C-labeled amino acids, ²H-glucose, ¹³C-labeled glucose, ²H-labeled organic molecules, ¹³C-labeled organic molecules, and ¹⁵N-labeled organic molecules.

“Protein precursor” refers to any organic or inorganic molecule or component thereof, wherein one or more atoms of which are capable of being incorporated into protein molecules in cell, tissue, organism, or other biological system, through the biochemical processes of the living system. Examples of protein precursors include, but are not limited to, amino acids, H₂O, CO₂, NH₃, and HCO₃.

“Isotope-labeled protein precursor” refers to a protein precursor that contains an isotope of an element that differs from the most abundant isotope of the element present in nature, cells, tissue, or organisms. The isotope label may include specific heavy isotopes of elements present in biomolecules, such as ²H, ¹³C, ¹⁵N, ¹⁸O, ³³S, ³⁴S, or may contain other isotopes of elements present in biomolecules such as ³H, ¹⁴C, ³⁵S, ¹²⁵I, ¹³¹I. Isotope-labeled protein precursors include, but are not limited to, ²H₂O, H₂¹⁸O, ¹⁵NH₃, ¹³CO₂, H¹³CO₃, ²H-labeled amino acids, ¹³C labeled amino acids, ¹⁵N labeled amino acids, ¹⁸O labeled amino acids, ³⁴S or ³³S labeled amino acids, ³H₂O ³H-labeled amino acids, and ¹⁴C labeled amino acids.

“Candidate agent” or “candidate drug” as used herein describes any molecule, e.g., proteins including biotherapeutics including antibodies and enzymes, small organic molecules including known drugs and drug candidates, polysaccharides, fatty acids, vaccines, nucleic acids, etc. that can be screened for activity as outlined herein. Candidate agents are evaluated in the present invention for a wide variety of reasons, including discovering potential therapeutic agents that affect molecular assemblage activity or assembly, and therefore potential disease states; for elucidating toxic effects of agents (e.g. environmental pollutants including industrial chemicals, pesticides, herbicides, etc.), drugs and drug candidates, food additives, cosmetics, etc.; drug discovery; as well as for elucidating new pathways associated with candidate agents (e.g. research into the side effects of drugs, etc.).

Candidate agents encompass numerous chemical classes. In one embodiment, the candidate agent is an organic molecule, preferably small organic compounds having a molecular weight of more than 100 and less than about 2,500 daltons. Particularly preferred are small organic compounds having a molecular weight of more than 100 and less than about 2,000 daltons, more preferably less than about 1500 daltons, more preferably less than about 1000 daltons, more preferably less than 500 daltons. Candidate agents comprise functional groups necessary for structural interaction with proteins, particularly hydrogen bonding, and typically include at least one of an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

“Drug leads” or “drug candidates” are herein defined as chemical entities or biological molecules that are being evaluated as potential therapeutic agents (drugs). “Drug agents” or “agents or “compounds” are used interchangeably herein and describe any composition of matter (e.g., chemical entity or biological factor) that is administered, approved or under testing as potential therapeutic agent or is a known therapeutic agent.

“Known drugs” or “known drug agents” or “already-approved drugs” refers to agents (e.g., chemical entities or biological factors) that have been approved for therapeutic use as drugs in human beings or animals in the United States or other jurisdictions. In the context of the present invention, the term “already-approved drug” includes a drug having approval for an indication distinct from an indication being tested for by use of the methods disclosed herein (“drug repurposing”). Using male infertility and fluoxetine as an example, the methods of the present invention allow one to test fluoxetine, a drug approved by the FDA (and other jurisdictions) for the treatment of depression, for effects on sperm motility and therefore for the treatment of male infertility; treating male infertility with fluoxetine is an indication not approved by FDA or other jurisdictions. In this manner, one can find new uses (in this example, a treatment for male infertility) for an already-approved drug (in this example, fluoxetine).

In addition, “already approved drugs” can be tested for effects on the assembly rates of molecular assemblages to elucidate drug pathways and potential undesirable side effects, that is, it can be tested within its indication.

Candidate agents are obtained from a wide variety of sources including libraries of synthetic or natural compounds. For example, numerous means are available for random and directed synthesis of a wide variety of organic compounds and biomolecules, including expression and/or synthesis of randomized oligonucleotides and peptides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

The candidate bioactive agents may be proteins. By “protein” herein is meant at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations. Peptide inhibitors of enzymes find particular use.

The candidate bioactive agents may be naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the systems described herein. Particularly preferred in this embodiment are libraries of bacterial, fungal, viral, and mammalian proteins, with the latter being preferred, and human proteins being especially preferred.

The candidate agents may be antibodies, a class of proteins. The term “antibody” includes full-length as well antibody fragments, as are known in the art, including Fab Fab2, single chain antibodies (Fv for example), chimeric antibodies, humanized and human antibodies, etc., either produced by the modification of whole antibodies or those synthesized de novo using recombinant DNA technologies, and derivatives thereof.

The candidate bioactive agents may be nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook, and peptide nucleic acids. Nucleic acids containing one or more carbocyclic sugars are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occuring nucleic acids and analogs may be made. The nucleic acids may be single stranded or double stranded, as specified, or contain portions of both double stranded or single stranded sequence, including restriction fragments, viruses, plasmids, chromosomes, etc. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine, etc. It should be noted in the context of the invention that nucleosides (ribose plus base) and nucleotides (ribose, base and at least one phosphate) are used interchangeably herein unless otherwise noted.

As described above generally for proteins, nucleic acid candidate bioactive agents may be naturally occurring nucleic acids, random and/or synthetic nucleic acids. For example, digests of procaryotic or eucaryotic genomes may be used as is outlined above for proteins. In addition, RNAis are included herein.

“Food additive” includes, but is not limited to, organoleptic agents (e.g., those agents conferring flavor, texture, aroma, and color), preservatives such as nitrosamines, nitrosamides, N-nitroso substances and the like, congealants, emulsifiers, dispersants, fumigants, humectants, oxidizing and reducing agents, propellants, sequestrants, solvents, surface-acting agents, surface-finishing agents, synergists, pesticides, chlorinated organic compounds, any chemical ingested by a food animal or taken up by a food plant, and any chemical leaching into (or otherwise finding its way into) food or drink from packaging material.

The term is meant to encompass those chemicals which are added into food or drink products at some step in the manufacturing and packaging process, or find their way into food by ingestion by food animals or uptake by food plants, or through microbial byproducts such as endotoxins and exotoxins (pre-formed toxins such as botulinin toxin or aflatoxin), or through the cooking process (such as heterocyclic amines, e.g., 2-amino-3-methyllimidazo[4,5-f]quinolone), or by leaching or some other process from packaging material during manufacturing, packaging, storage, and handling activities.

“Industrial chemical” includes, but is not limited to, volatile organic compounds, semi-volatile organic compounds, cleaners, solvents, thinners, mixers, metallic compounds, metals, organometals, metalloids, substituted and non-substituted aliphatic and acyclic hydrocarbons such as hexane, substituted and non-substituted aromatic hydrocarbons such as benzene and styrene, halogenated hydrocarbons such as vinyl chloride, aminoderivatives and nitroderivatives such as nitrobenzene, glycols and derivatives such as propylene glycol, ketones such as cyclohexanone, aldehydes such as furfural, amides and anhydrides such as acrylamide, phenols, cyanides and nitriles, isocyanates, and pesticides, herbicides, rodenticides, and fungicides.

“Environmental pollutant” includes any chemical not found in nature or chemicals that are found in nature but artificially concentrated to levels exceeding those found in nature (at least found in accessible media in nature). So, for example, environmental pollutants can include any of the non-natural chemicals identified as an occupational or industrial chemical yet found in a non-occupational or industrial setting such as a park, school, or playground. Alternatively, environmental pollutants may comprise naturally occurring chemicals such as lead but at levels exceeding background (for example, lead found in the soil along highways deposited by the exhaust from the burning of leaded gasoline in automobiles). Environmental pollutants may be from a point source such as a factory smokestack or industrial liquid discharge into surface or groundwater, or from a non-point source such as the exhaust from cars traveling along a highway, the diesel exhaust (and all that it contains) from buses traveling along city streets, or pesticides deposited in soil from airborne dust originating in farmlands. As used herein, “environmental contaminant” is synonymous with “environmental pollutant.”

“Partially purifying” refers to methods of removing one or more components of a mixture of other similar compounds. For example, “partially purifying a protein” refers to removing one or more proteins from a mixture of one or more proteins. “Isolating” refers to separating one compound from a mixture of compounds. For example, “isolating a protein” refers to separating one specific protein from all other proteins in a mixture of one or more proteins.

“Living system” includes, but is not limited to, cells (including primary cells), cell lines (including cell lines of healthy and diseased cells), plants, bacteria (particularly bacteria with cilia and/or flagella) and animals, particularly mammals and particularly human. Suitable cells include, but are not limited to, tumor cells of all types (particularly melanoma, myeloid leukemia, carcinomas of the lung, breast, ovaries, colon, kidney, prostate, brain, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, myocytes, fibroblasts, neurons, glial cells, pancreatic cells, intestinal epithelial cells, lymphocytes, erythrocytes, adipocytes, myocytes, fibroblasts, neurons, duct cells and acinar cells of the pancreas, gastrointestinal epithelial cells, leukocytes, lymphocytes, erythrocytes, keratinocytes, pulmonary epithelial cells, cervical epithelial cells, endometrial cells, ovarian cells (e.g., ovarian stromal cells), breast epithelial cells, melanocytes, melanoma cells, prostate epithelial cells, bladder epithelial cells, astrocytes, sperm cells, microbial cells and any other cell-type that can be maintained alive and functional in vitro. Microbial and plant cells can also be used. In particular, when the molecular assemblages comprise microtubules, cells that contain cilia (such as endothelial cells lining the respiratory tract of mammals such as humans) and/or flagella, such as sperm cells and flagellated bacterial cells, can be evaluated as is more fully described below.

In one embodiment, the cells may be genetically engineered, that is, contain exogeneous nucleic acid.

The cell may be collected from a multicellular organism and cultured or may be purchased from a commercial source such as the American Type Culture Collection and propagated as a cell line using techniques well known in the art. Suitable cell lines include, but are not limited to, cell lines made from any of the above-mentioned cells, as well as established cell lines such as Suitable cells also include known research cells, including, but not limited to, Jurkat T cells, NIH3T3 cells, CHO, Cos, etc. See the ATCC cell line catalog, hereby expressly incorporated by reference. Suitable mammals include, but are not limited to, any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are included within the definition herein. Living systems can either be control systems, which are free from perturbation such as treatment with candidate agents or free of disease or risk of disease, or systems under evaluation. “Living system” includes individual subjects, including human patients.

A “biological sample” encompasses any sample obtained from a living system, including subcellular components, cells, tissues, or an organism. The sample may be solid in nature. The definition also encompasses fluid, including liquid, samples of biological origin, that are accessible from an organism through sampling by minimally invasive or non-invasive approaches (e.g., urine collection, needle aspiration, breast fluid collection from breast ductal lavage, skin scraping, semen collection, vaginal secretion collection, nasal secretion collection, sputum collection, stool collection, and other procedures involving minimal risk, discomfort or effort). The definition also includes samples that have been manipulated in any way after their procurement, such as by treatment with reagents, solubilization, or enrichment for certain components, such as assemblages including proteins, lipids, carbohydrates, or organic metabolites. The term “biological sample” also encompasses a clinical sample such as serum, plasma, other biological fluid, or tissue samples, and also includes cells in culture, cell supernatants and cell lysates.

“Biological fluid” refers to, but is not limited to, urine, edema fluid, saliva, lacrimal fluid, inflammatory exudates, synovial fluid, abscess, empyema or other infected fluid, sweat, pulmonary secretions (sputum), seminal fluid, feces, bile, intestinal secretions, vaginal secretions, or any other biological fluid found in spaces external to the body (e.g., luminal or integumentary spaces).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nominal mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as Mo; for most organic molecules, this is the species containing all ¹²C, ¹ H, ¹⁶O, ¹⁴N, etc. Other mass isotopomers are distinguished by their mass differences from M₀ (M₁, M₂, etc.). For a given mass isotopomer, the location or position of isotopes within the molecule is not specified and may vary (e.g. “positional isotopomers” are not distinguished).

“Mass isotopomer envelope” refers to the set of mass isotopomers associated with a molecule or ion fragment.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonomously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as mo and the masses of other mass isotopomers are identified by their mass differences from m₀ (m₁, m₂, etc.).

“Isotopically perturbed” refers to the state of an element or molecule that results from the explicit incorporation of an element or molecule with a distribution of isotopes that differs from the distribution found in nature, whether a naturally less abundant isotope is present in excess (enriched) or in deficit (depleted).

By “molecule of interest” is meant any molecule (polymer and/or monomer), including but not limited to, amino acids, carbohydrates, fatty acids, peptides, sugars, lipids, nucleic acids, polynucleotides, glycosaminoglycans, polypeptides, or proteins that self-assemble into a molecular assemblage of the invention. In the context of the present invention, a “molecule of interest” may be a “biomarker” of disease and its flux rate, relative to the flux rate of an unexposed or otherwise healthy subject (e.g., control subject), may represent clinically non-observant or subtle pathophysiological occurrences in a subject of interest that may be predictive of future disease or injury in the subject of interest. In this manner, comparing the flux rates of one or more biomarkers of interest in a subject of interest with the flux rates of one or more biomarkers of interest in a control subject, will find use in diagnosing the subject of interest with, or evaluating or quantifying the subject of interest's risk in acquiring, a disease of interest. Moreover, such information will find use in establishing a prognosis for a subject of interest having a disease of interest, monitoring the progression of a disease of interest in a subject of interest, or evaluating the therapeutic efficacy of a treatment regimen in a subject of interest having a disease of interest.

By “subject of interest” is meant a human, animal or cell having a disease of interest, having some level of risk in acquiring a disease of interest, or being evaluated for the effects of a candidate agent.

By “control subject” is meant a human or animal not having the disease of interest, not having some level of risk in acquiring the disease of interest, or not being evaluated for a candidate agent effect.

“Monomer” refers to a chemical unit that combines during the synthesis of a polymer and which is present two or more times in the polymer.

“Polymer” refers to a molecule synthesized from and containing two or more repeats of a monomer.

“Isotope-labeled substrate” includes any isotope-labeled precursor molecule that is able to be incorporated into a molecule of interest in a living system. Examples of isotope-labeled substrates include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃. “Deuterated water” refers to water incorporating one or more ²H isotopes. “Labeled glucose” refers to glucose labeled with one or more ²H isotopes. Specific examples of labeled glucose or ²H-labeled glucose include [6,6-²H₂]glucose, [1-²H₁]glucose, and [1,2,3,4,5,6-²H₇] glucose.

“Administer[ed]” includes a living system exposed to a compound, including candidate agents and labeled substrates, Such exposure can be from, but is not limited to, topical application, oral ingestion, inhalation, subcutaneous injection, intraperitoneal injection, intravenous injection, and intraarterial injection, in animals or other higher organisms. Administration to cells, tissue culture or cell lines can be adding the compound to the growth media.

An “individual” is a vertebrate, preferably a mammal, more preferably a human.

“At least partially identified” in the context of drug discovery and development means at least one clinically relevant pharmacological characteristic of a drug agent (e.g., a “compound”) has been identified using one or more of the methods of the present invention. This characteristic may be a desirable one, for example, increasing or decreasing molecular flux rates through a metabolic pathway that contributes to a disease process, altering signal transduction pathways or cell surface receptors that alter the activity of metabolic pathways relevant to a disease, inhibiting activation of an enzyme and the like. Alternatively, a pharmacological characteristic of a drug agent may be an undesirable one for example, the production of one or more toxic effects. There are a plethora of desirable and undesirable characteristics of drug agents well known to those skilled in the art and each will be viewed in the context of the particular drug agent being developed and the targeted disease. Of course, a drug agent can be more than at least partially identified when, for example, when several characteristics have been identified (desirable or undesirable or both) that are sufficient to support a particular milestone decision point along the drug development pathway. Such milestones include, but are not limited to, pre-clinical decisions for in vitro to in vivo transition, pre-IND filing go/no go decision, phase I to phase II transition, phase II to phase III transition, NDA filing, and FDA approval for marketing. Therefore, “at least partially” identified includes the identification of one or more pharmacological characteristics useful in evaluating a drug agent in the drug discovery/drug development process. A pharmacologist or physician or other researcher may evaluate all or a portion of the identified desirable and undesirable characteristics of a drug agent to establish its therapeutic index. This may be accomplished using procedures well known in the art.

“Manufacturing a drug agent” in the context of the present invention includes any means, well known to those skilled in the art, employed for the making of a drug agent product. Manufacturing processes include, but are not limited to, medicinal chemical synthesis (e.g., synthetic organic chemistry), combinatorial chemistry, biotechnology methods such as hybridoma monoclonal antibody production, recombinant DNA technology, and other techniques well known to the skilled artisan. Such a product may be a final drug agent that is marketed for therapeutic use, a component of a combination product that is marketed for therapeutic use, or any intermediate product used in the development of the final drug agent product, whether as part of a combination product or a single product. “Manufacturing drug agent” is synonymous with “manufacturing a compound.”

By “biomarker” is meant a biochemical measurement from the organism which is useful or potentially useful for measuring the initiation, progression, severity, pathology, aggressiveness, grade, activity, disability, mortality, morbidity, disease sub-classification or other underlying pathogenic or pathologic feature of one or more diseases. The concept of a biomarker also includes a physical measurement on the body, such as blood pressure, which is useful for measuring the initiation, progression, severity, pathology, aggressiveness, grade, activity, disability, mortality, morbidity, disease sub-classification or other underlying pathogenic or pathologic feature of one or more diseases. The concept of a biomarker also includes a pharmacological or physiological measurement which is used to predict a toxicity event in an animal or a human. A biomarker may be the target for monitoring the outcome of a therapeutic intervention (e.g., the target of a drug agent).

By “evaluate” or “evaluation” or “evaluating,” in the context of the present invention, is meant a process whereby the activity, toxicity, relative potency, potential therapeutic value and/or efficacy, significance, or worth of a chemical entity, biological factor, combination of chemical entities, or combination of biological factors is determined through appraisal and study, usually by means of comparing experimental outcomes to established standards and/or conditions. The term embraces the concept of providing sufficient information for a decision-maker to make a “go/no go” decision on a chemical entity or biological factor (or combinations of chemical entities or combinations of biological factors) to proceed further in the drug development process. A “go/no go” decision may be made at any point or milestone in the drug development process including, but not limited to, any stage within pre-clinical development, the pre-clinical to Investigational New Drug (IND) stage, the Phase I to Phase II stage, the Phase II to more advanced phases within Phase II (such as Phase IIb), the Phase II to Phase III stage, the Phase III to the New Drug Application (NDA) or Biologics License Application (BLA) stage, or stages beyond (such as Phase IV or other post-NDA or post-BLA stages). The term also embraces the concept of providing sufficient information to select “best-in-breed” (or “best-of-breed”) in a class of compounds (chemical entities, biologics).

By “characterize,” “characterizing,” or “characterization,” in the context of the present invention is meant an effort to describe the character or quality of a chemical entity or combination of chemical entities. As used herein, the term is nearly equivalent to “evaluate,” yet lacks the more refined aspects of “evaluate,” in which to “evaluate” a drug includes the ability to make a “go/no go” decision (based on an assessment of therapeutic value) on proceeding with that drug or

By “condition” or “medical condition” is meant the physical status of the body as a whole or of one of its parts. The term is usually used to indicate a change from a previous physical or mental status, or an abnormality not recognized by medical authorities as a disease or disorder. Examples of “conditions” or “medical conditions” include obesity and pregnancy.

By “proliferative disorder” or “proliferative disease” is meant any disease that is characterized by uncontrolled cell proliferation such as cancer. Non-malignant diseases or disorders characterized by uncontrolled or hypercellular proliferation, such as psoriasis, are also proliferative disorders or proliferative diseases within the meaning of the

### METHODS OF THE INVENTION

The present invention is directed to methods of determining the dynamics (e.g., self-assembly and disassembly rates; synthesis and breakdown rates) of a plurality of self-assembling systems of biological molecules in a living system (e.g. cell, tissue or organism). First, one or more isotope-labeled substrates (sometimes referred to herein as “precursors”) are administered to the living system for at least a first period of time sufficient to be incorporated into a plurality of subunits of self-assembling systems of biological molecules. The labeled molecular assemblages are obtained from the living system in a variety of ways, for example cell fractionation, and the amount of label is usually quantified. In addition, “unincorporated” labeled substrates can also be quantified; for example using mass spectrometry as outlined herein.

As outlined above, in this manner, the dynamics of microtubules can be determined by measuring and comparing, over specific time intervals, the isotopic content and/or pattern or the rate of change of the isotopic content and/or pattern in the targeted subunit or molecular assemblage (e.g., tubulin dimers and microtubule polymers), for example by using mass spectrometry or other analytical techniques known in the art. The relationship between the isotopic content and/or pattern or the rate of change of the isotopic content and/or pattern in the molecular assemblage (e.g., a microtubule) to the isotopic content and/or pattern or rate of change in the isotopic content and/or pattern in the unassembled subunits may be particularly informative. (It should be noted, however, that not all systems analysis requires the quantification or evaluation of the “unincorporated” or “free” substrate.) The dynamics of microtubule assembly and disassembly (polymerization and depolymerization) can then be calculated. In a like manner, the dynamics of any self-assembling system of biological molecules, such as actin filaments (e.g., microfilaments) in the cytoskeleton, mutant hemoglobin in sickle-cell erythrocytes, amyloid-beta fibrils in the brain, tau filaments or aggregates (neurofibrillary tangles) in the brain, □-synuclein filaments or aggregates (Lewy bodies or Lewy neurites) in the brain, prion aggregates or plaques in the brain, fibrin assemblages in blood clots, galactocerebroside aggregates in the myelin lamella, phospholipids aggregates (or diminished aggregates) in the myelin lamella, cardiolipin aggregates in the inner membrane of the mitochondrion, cholesterol aggregates in the plasma membrane, or phospholipid aggregates in the plasma membrane, myelin lamellae, or the membrane of any subcellular organelle, can be calculated.

Alternatively, radiolabeled substrates are contemplated for use in the present invention wherein the radiolabeled substrates are incorporated into tubulin dimers, which are then incorporated into microtubule polymers. In this manner, the dynamics of microtubules can be determined by measuring radioactivity present in the tubulin dimers and microtubule polymers by using techniques known in the art such as scintillation counting. The dynamics of microtubule polymers are then calculated, using methods known in the art. Radiolabeled substrates can also be used for other self-assembling systems of biological molecules in a like manner.

In another embodiment, the dynamics of microtubules are measured from the assembly and disassembly (polymerization and depolymerization) of microtubules in a living organism prior to, and after, exposure to one or more candidate agents, to evaluate toxicity. As will be appreciated by those in the art, a variety of suitable classes of candidate agents may be tested for toxicity, including, but not limited to, industrial or occupational chemicals, cosmetics, food additives, environmental pollutants, drugs and drug candidates, etc. In a like manner, the dynamics of assembly and disassembly of any self-assembling systems of biological molecules can be measured prior to, during and/or after, exposure to one or more candidate

Alternatively, exposure of a living system to candidate agents and the dynamics of microtubules are compared to the dynamics of microtubules from an unexposed living system of the same species to evaluate toxicity (e.g. the use of multiple living systems to evaluate toxicity).

As for all the self assembling systems of the present invention, comparisons can also be made at different time points or for different time periods, different doses of candidate agents, different combinations of candidate agents, different “pulse-chase” experiments, or combinations thereof. For example, dose curves can be run, or dose time curves, or matrices thereof.

In an additional embodiment of the invention, microtubule polymerization and depolymerization is evaluated in sperm cells and/or sperm formation (e.g., testes tissue) for a variety of reasons, including the development of drugs to increase sperm mobility (fertility) or decrease sperm mobility (birth control), or for the evaluation of drugs and drug candidates on sperm mobility and/or formation, amongst other reasons.

In a further embodiment, eukaryotic cells with cilia, such as those lining the respiratory and gastrointestinal tracts, can be used as well, as microtubule formation is crucial to cilia formation and action.

In another embodiment, the living systems are flagellated bacterial cells. Many bacterial strains utilize flagella for motility and/or infectivity, and thus evaluating the dynamics of microtubule polymerization and depolymerization can be used in drug development such as antibiotics.

In another embodiment of the invention, the dynamics of assembly and disassembly in self-assembling biological systems other than tubulin can be measured. One such example of a self-assembling biological system is Aβ fibrils or plaques formed from A□ peptides (Aβ₁₋₄₀ or Aβ₁₋₄₂, which are derived by proteolytic processing from Amyloid Precursor Protein (APP)) in the brains of patients with Alzheimer's disease or the brains in animal models of Alzheimer's disease. The dynamics of amyloid fibrillogenesis is of central interest to pathogenesis, progression, and potential therapeutic efficacy of interventions in Alzheimer's disease. Dynamics of Aβ fibrils are measurable by use of the methods of the invention disclosed herein. In one embodiment, a stable isotope-labeled substrate is administered to an animal model of Alzheimer's disease, such as a transgenic mouse expressing or overexpressing APP, or to a patient with Alzheimer's disease. The incorporation of said stable isotopic label into Aβ plaques (fibrils) and into Aβ peptides (or peptides co-synthesized in APP) are then measured by methods known in the art. In this manner, the dynamics of Aβ plaques (fibrils) can be determined by measuring and comparing, over specific time intervals, the isotopic content and/or pattern or the rate of change of the isotopic content and/or pattern in the targeted self-assembling molecules (e.g., Aβ fibrils or plaques) and free Aβ peptides, by use of mass spectrometry or other analytic techniques known in the art. The relationship between labeling (e.g., the isotopic content and/or pattern or the rate of change of the isotopic content and/or pattern) in the aggregated form (the Aβ fibrils or plaque) and the unaggregated subunits (the free Aβ peptides or peptides co-synthesized in APP) may be particularly informative.

The dynamics of assembly and disassembly of Aβ fibrils or plaque can thereby be calculated, by use of calculation methods known in the art.

As described for the example of microtubules, the methods of the invention allow for the comparison between the dynamics of Aβ fibrils or plaque assembly and disassembly measured from living systems that have been exposed to one or more compounds to the dynamics of Aβ fibrils or plaque measured from non-exposed living systems. Alternatively, dynamics of Aβ fibrils or plaque can be calculated from living systems prior to exposure to one or more compounds and then the rates may be calculated in the same living systems after exposure to said one or more compounds and then compared.

In other embodiments of the invention, the dynamics of assembly and disassembly in other self-assembling biological systems are measured. An example of such self-assembling biological systems includes sickled hemoglobin aggregates formed from mutant hemoglobins (e.g., hemoglobin S) in erythrocytes of animals or patients with sickle cell disease. By analogy to the self-assembling systems described above, incorporation of isotopically-labeled substrates into sickled hemoglobin aggregates are thereby determined, based on the isotopic content and/or pattern or the rate of change of the isotopic content and/or pattern in the aggregated molecules (e.g., sickled hemoglobin aggregates) and in free hemoglobin protein. The relationship between labeling in the aggregated form (sickled hemoglobin) and the unaggregated subunits (free hemoglobin) may be particularly informative. The dynamics of assembly and disassembly of sickled hemoglobin can thereby be calculated, using calculation methods known in the art. Effects of exposure to one or more compounds on the dynamics of sickled hemoglobin aggregates can thereby be determined by the methods described herein.

Another example of a self-assembling biological system is prions, which are implicated in Creutzfeldt-Jakob disease (CJD), kuru, scrapies, and bovine spongiform encephalopathy (BSE). Free prion protein (PrP) subunit, normally present in the brain, may precipitate into aggregates of rod-shaped particles after nucleation induced by infectious prion aggregates. The conversion of normal PrP to pathologic aggregates involves conformational change and polymerization. By analogy to the self-assembling systems described, supra (and described more fully, infra), incorporation of isotopically-labeled substrates into prion aggregates and free PrP are measured and compared over specific time intervals. The dynamics of assembly and disassembly of prion aggregates are thereby determined, based on the isotopic content and/or pattern or the rate of change of the isotopic content and/or pattern in the aggregated form (the prion rod-shaped particles) and the unaggregated subunits (free PrP), measured by the use of mass spectrometry or other analytic techniques known in the art. The relationship between labeling (e.g., the isotopic content and/or pattern or the rate of change of the isotopic content and/or pattern) in the aggregated form (the prion fibrils) and the unaggregated subunits (free PrP) may be particularly informative. The dynamics of assembly and disassembly of prion fibrils can thereby be calculated, by use of calculation methods known in the art. Effects of exposure to one or more compounds on the dynamics of assembly and disassembly of prion fibrils can thereby be determined by the methods of the invention described herein.

Another example of a self-assembling biological system is actin. Free actin exists as a globular monomer called G-actin. Monomeric G-actin self assembles into a filamentous polymer called F-actin. The F-actin polymers comprise the cellular cytoskeleton. The cytoskeleton is dynamic, e.g., actin microfilaments are constantly shrinking or growing in length, and bundles and meshworks of microfilaments are continually forming and dissolving. The polymerization and depolymerization of filamentous actin is important in many cell functions including cytokinesis. By analogy to the self-assembly of microtubules and other systems described, supra and infra, incorporation of isotopically-labeled substrates into F-actin filaments and free G-actin are measured and compared over specific time intervals. The dynamics of assembly and disassembly of actin filaments are thereby determined, based on the isotopic content and/or pattern or the rate of change of the isotopic content and/or pattern in the filamentous form (the F-actin polymer) and the free subunits (G-actin monomers), measured by the use of mass spectrometry or other analytic techniques known in the art. The relationship between labeling (e.g., the isotopic content and/or pattern or the rate of change of the isotopic content and/or pattern) in the filamentous form and the free form may be particularly informative. The dynamics of assembly and disassembly of actin filaments can thereby be calculated, by use of calculation methods known in the art. Effects of exposure to one or more compounds on the dynamics of assembly and disassembly of actin filaments can thereby be determined by the methods of the invention described herein.

Another example of such self-assembling biological systems includes the formation of blood clots. Blood clots are formed when free fibrin monomers self-assemble into fibrin fibers. By analogy to the self-assembling systems described above, incorporation of isotopically-labeled substrates into fibrin fibers (e.g., blood clots) are thereby determined, based on the isotopic content and/or pattern or the rate of change of the isotopic content and/or pattern in the fibrin fibers (e.g., a blood clot) and in free fibrin. The relationship between labeling in the aggregated form (blood clot) and the unaggregated subunits (free fibrin or its precursor, fibrinogen) may be particularly informative. The dynamics of assembly and disassembly of fibrin fibers (e.g., blood clots) can thereby be calculated, using calculation methods known in the art. Effects of exposure to one or more compounds on the dynamics of fibrin fibers can thereby be determined by the methods described herein.

In another embodiment of the invention, the measurement of microtubule dynamics in post-mitotic cells (e.g., non-dividing cells such as differentiated neurons), which does not reflect the proliferation rate of said cells, allows for the measurement of other important biological processes such as cellular toxicity (e.g., axonal dysfunction), as more fully

Changes in the dynamics of self-assembling systems of biological molecules can be elicited by candidate agents, for example, known drugs, drug candidates, drug leads (or combinations thereof), or industrial chemicals such as pesticides, herbicides, plastics, and the like, or cosmetics, or food additives.

At least one isotope-labeled substrate molecule is administered to a living system for a period of time sufficient to be incorporated in vivo (or intracellularly if the living system is a cultured cell such as a cell line or bacteria) into one or more subunits of self-assembling systems of biological molecules (e.g., tubulin dimers, Aβ protein, actin, tau protein, α-synuclein, mutant hemoglobin, fibrin, free prion protein, galactocerebroside, cardiolipin, cholesterol, phosphatidylserine, phosphatidylcholine, phosphatidylinositol, and phosphatidylethanolamine) that are then incorporated into molecular assemblages. In one embodiment, the isotope-labeled substrate molecule is labeled with a stable isotope (e.g., non-radioactive isotope). In another embodiment, the isotope-labeled substrate molecule is labeled with a radioactive isotope. In yet another embodiment, both stable and radioactive isotopes are used to label one or more isotope-labeled substrate molecules.

The labeled subunits and/or labeled molecular assemblages are obtained by biochemical isolation procedures from the living system, and identified by mass spectrometry or by other analytical techniques known in the art. The relative and absolute abundances of the ions within the mass isotopomeric envelope corresponding to each identified subunit or molecular assemblage (e.g., the isotopic content and/or pattern of the molecule or the rate of change of the isotopic content and/or pattern of the molecule) are quantified. In one embodiment, the relative and absolute abundances of the ions within the mass isotopomeric envelope corresponding to each identified subunit or molecular assemblage are quantified by mass spectrometry. The dynamics of self-assembling systems of biological molecules are then calculated by use of equations known in the art and discussed, infra. The calculated dynamics are compared in the presence or absence of exposure to one or more candidate agents or combinations of candidate agents, or in response to different levels of exposure to one or more candidate agents or combinations of agents.

In this manner, changes in the dynamics of self-assembling systems of biological molecules are measured and quantified and related to disease diagnosis; disease prognosis; therapeutic efficacy of administered candidate agents; and/or toxic effects of candidate agents, as well as for drug discovery.

### Administering Isotope-Labeled Precursor(s)

As a first step in the method of the invention, isotope-labeled precursors are administered.

### Administering an Isotope-Labeled Precursor Molecule

### Labeled Precursor Molecules

### Isotope Labels

The first step in measuring molecular flux rates involves administering an isotope-labeled precursor molecule to a living system. The isotope-labeled precursor molecule may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems.

In one embodiment, the isotope label is ²H.

Precursor Molecules

The precursor molecule may be any molecule having an isotope label that is incorporated into the “monomer” or “subunit” of interest, or it can be the monomer itself. Isotope labels may be used to modify all precursor molecules disclosed herein to form isotope-labeled precursor molecules.

The entire precursor molecule may be incorporated into one or more subunits (e.g. proteins, phospholipids, cholesterol (e.g., α□-tubulin, β-tubulin)). Alternatively, a portion of the precursor molecule may be incorporated into the subunits.

### Protein Precursors

A protein precursor molecule may be any protein precursor molecule known in the art. These precursor molecules include, but are not limited to, CO₂, NH₃, glucose, lactate, H₂O, acetate, and fatty acids.

Precursor molecules of proteins may also include one or more amino acids. The precursor may be any amino acid. The precursor molecule may be a singly or multiply deuterated amino acid. For example, the precursor molecule may be one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted or diluted with non-labeled amino acids. All isotope-labeled precursors may be purchased commercially, for example, from Cambridge Isotope Labs (Andover, Mass.).

Protein precursor molecules may also include any precursor for post-translationally or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; precursors of glycosylation and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non essential amino acid is known—e.g., 4 in alanine, 2 in glycine, etc.

The precursor molecule for proteins may be water (e.g., heavy water). The hydrogen atoms on C—H bonds are the hydrogen atoms on amino acids that are useful for measuring protein synthesis from ²H₂O since the O—H and N—H bonds of proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—e.g., that the protein is newly synthesized. Analytically, the amino acid derivative used must contain all the C—H bonds but must remove all potentially contaminating N—H and O—H bonds.

Hydrogen atoms (e.g., deuterium ortritium) from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the □-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins.

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor □-ketoglutarate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histidine, the hydroxyl group in hydroxyproline or hydroxylysine, and others. Other amino acid synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme-catalyzed reactions. Incorporation of labeled oxygen into amino acids is known to one of skill in the art. Oxygen atoms may also be incorporated into amino acids from ¹⁸O₂ through enzyme-catalyzed reactions (including hydroxyproline, hydroxylysine or other post-translationally modified amino acids).

Hydrogen and oxygen labels from labeled water also may be incorporated into amino acids through post-translational modifications. In one embodiment, the post-translational modification already may include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange-labeled hydrogens from body water, either before or after post-translational modification step (e.g., methylation, hydroxylation, phosphorylation, prenylation, sulfation, carboxylation, acetylation, glycosylation, or other known post-translational modifications).

Protein precursors that are suitable for administration into a subject include, but are not limited to, H₂O, CO₂, NH₃ and HCO₃, in addition to the standard amino acids found in proteins as described, supra.

### Water as a Precursor Molecule

Water is a precursor of proteins (and DNA and other biomolecules such as lipids). As such, labeled water (e.g., heavy water) may serve as a precursor in the methods taught herein.

Labeled water may be readily obtained commercially. For example, ²H₂O may be purchased from Cambridge Isotope Labs (Andover, Mass.), and ³H₂O may be purchased, e.g., from New England Nuclear, Inc. In general, ²H₂O (and H₂¹⁸O) is non-radioactive and thus, presents fewer toxicity concerns than radioactive ³H₂O. ²H₂O may be administered, for example, as a percent of total body water, e.g., 1% of total body water consumed (e.g., for 3 liters water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved relatively inexpensively using the techniques of the invention. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. One of the present inventors has discovered that as long as rapid changes in body water enrichment are prevented (e.g., by initial administration in small, divided doses), high body water enrichments of ²H₂O can be maintained with no toxicities. For example, the low cost of commercially available ²H₂O allows long-term maintenance of enrichments in the 1-5% range at relatively low expense (e.g., calculations reveal a lower cost for 2 months of labeling at 2% ²H₂O enrichment, and thus 7-8% enrichment in the alanine precursor pool, than for 12 hours of labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope, like ²H, is not toxic, and does not present a significant health risk as a result.

### Lipid Precursors

Labeled precursors of lipids may include any precursor in lipid biosynthesis.

The precursor molecules of lipids may be CO₂, NH₃, glucose, lactate, H₂O, acetate, and fatty acids.

The precursor may also include labeled water, preferably ²H₂O, which is a precursor for fatty acids, glycerol moiety of acyl-glycerols, cholesterol and its derivatives; ¹³C or ²H-labeled fatty acids, which are precursors for triglycerides, phospholipids, cholesterol ester, coamides and other lipids; ¹³C- or ²H-acetate, which is a precursor for fatty acids and cholesterol; ¹⁸O₂, which is a precursor for fatty acids, cholesterol, acyl-glycerides, and certain oxidatively modified fatty acids (such as peroxides) by either enzymatically catalyzed reactions or by non-enzymatic oxidative damage (e.g., to fatty acids); ¹³C- or ²H-glycerol, which is a precursor for acyl-glycerides; ¹³C- or ²H-labeled acetate, ethanol, ketone bodies or fatty acids, which are precursors for endogenously synthesized fatty acids, cholesterol and acylglycerides; and ²H or ³C-labeled cholesterol or its derivatives (including bile acids and steroid hormones). All isotope labeled precursors may be purchased commercially, for example, from Cambridge Isotope Labs (Andover, Mass.).

Complex lipids, such as glycolipids, phospholipids, and cerebrosides (including galactocerebroside), can also be labeled from precursors, including ²H₂O, which is a precursor for the sugar-moiety of cerebrosides (including, but not limited to, N-acetylgalactosamine, N-acetylglucosamine-sulfate, glucuronic acid, and glucuronic acid-sulfate), the fatty acyl-moiety of cerebrosides and the sphingosine moiety of cerebrosides; ²H- or ¹³C-labeled fatty acids, which are precursors for the fatty acyl moiety of cerebrosides, glycolipids, phospholipids and other derivatives.

The precursor molecule may be or include components of lipids.

### Modes of Administering Precursors

Modes of administering the one or more isotope-labeled precursors may vary, depending upon the absorptive properties of the isotope-labeled precursor and the specific biosynthetic pool into which each compound is targeted. Precursors may be delivered to multicellular organisms, including experimental animals (e.g., various animal models of proliferative diseases or healthy animals) and humans, directly for in vivo analysis. In addition, precursors may be administered in vitro to living cells.

Generally, an appropriate mode of administration is one that produces a steady state level of precursor within the biosynthetic pool and/or in a reservoir supplying such a pool for at least a transient period of time. Intravascular (such as intravenous) or oral routes of administration are commonly used to administer such precursors to organisms, including humans. Other routes of administration, such as subcutaneous or intramuscular administration, optionally when used in conjunction with slow release precursor compositions, are also appropriate. Compositions for injection are generally prepared in sterile pharmaceutical excipients as is well known in the art. Adding the components (either precursors or candidate agents) to cell media can be done for in vitro systems.

Obtaining a plurality of proteins and other subunits of self-assembling systems of biological molecules

In practicing the method of the invention, in one aspect, proteins and subunits of other self-assembling systems of biological molecules (e.g., phospholipids, cholesterol) are obtained from a living system according to methods known in the art.

A plurality of molecular assemblages and/or free subunits of self-assembling systems of biological molecules are acquired from the living system. The one or more biological samples may be obtained, for example, by blood draw, urine collection, biopsy, or other methods known in the art. The one or more biological samples may be one or more biological fluids. The proteins or subunits of other self-assembling systems of biological molecules may also be obtained from specific organs or tissues (or any cell type comprising a tissue or organ), such as muscle, liver, brain, adrenal tissue, prostate tissue, endometrial tissue, blood, skin, breast tissue, or any other tissue or cell of the body (e.g., epithelial cells of any tissue). Proteins or subunits of other self-assembling systems of biological molecules may be obtained from a specific group of cells, such as tumor cells or other growing or non-growing cells. Proteins or subunits of other self-assembling systems of biological molecules also may be obtained, and optionally partially purified or isolated, from the biological sample using standard biochemical methods known in the art.

The frequency of biological sampling can vary depending on different factors. Such factors include, but are not limited to, ease and safety of sampling, synthesis and breakdown/removal rates of the proteins or other subunits of self-assembling systems of biological molecules, and the half-life of a therapeutic chemical agent or biological agent (e.g., a therapeutic compound) administered to a cell, animal, or human.

Proteins and subunits of other self-assembling systems of biological molecules may be partially purified and/or isolated from one or more biological samples, depending on the assay requirements. In general, molecular assemblages and/or subunits may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, fast performance liquid chromatography (FPLC), chemical extraction, thin layer chromatography, gas chromatography, and chromatofocusing. For example, some proteins may be purified using a standard antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the assay and components of the system. In some instances no purification will be necessary.

In another embodiment, the proteins or subunits of other self-assembling systems of biological molecules (e.g., galactocerebroside or phospholipids and/or cholesterol for plasma or organelle membranes or the myelin lamella) may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the proteins or subunits of other self-assembling systems of biological molecules. The proteins or subunits of other self-assembling systems of biological molecules also may be partially purified, or optionally, isolated, by conventional purification methods including HPLC, FPLC, gas chromatography, gel electrophoresis, and/or any other methods of separating chemical and/or biochemical compounds known to those skilled in the art.

### ANALYSIS

### Mass Spectrometry

Isotopic enrichment in proteins or subunits of other self-assembling systems of biological molecules can be determined by various methods known in the art such as mass spectrometry, including but not limited to, gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS.

Mass spectrometers convert molecules such as proteins or lipids into rapidly moving gaseous ions and separate them on the basis of their mass-to-charge ratios. The distributions of isotopes or isotopologues of ions, or ion fragments, may thus be used to measure the isotopic enrichment in a plurality of proteins or phospholipid or cholesterol molecules.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass analyzers may be coupled (MS/MS) first to separate precursor ions, then to separate and measure gas phase fragment ions. These instruments generate an initial series of ionic fragments of a protein or phospholipid or cholesterol or galactocerebroside, and then generate secondary fragments of the initial ions.

Different ionization methods are also known in the art. One key advance has been the development of techniques for ionization of large, non-volatile macromolecules including proteins, phospholipids, galactocerebroside, and cholesterol. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

In general, in order to determine a baseline mass isotopomer frequency distribution for the protein or phospholipid or galactocerebroside or cholesterol, such a sample is taken before infusion of an isotopically labeled precursor. Such a measurement is one means of establishing in the cell, tissue or organism, the naturally occurring frequency of mass isotopomers of the protein or phospholipids or galactocerebroside or cholesterol. When a cell, tissue or organism is part of a population of subjects having similar environmental histories, a population isotopomer frequency distribution may be used for such a background measurement. Additionally, such a baseline isotopomer frequency distribution may be estimated, using known average natural abundances of isotopes. For example, in nature, the natural abundance of ¹³C present in organic carbon is 1.11%. Methods of determining such isotopomer frequency distributions are discussed below. Typically, samples of the protein or phospholipids or cholesterol are taken prior to and following administration of an isotopically

### Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the invention.

Calculating Labeled: Unlabeled Proportion of Proteins or Subunits of other Self-Assembling Systems of Biological Molecules

The proportion of labeled or unlabeled proteins and/or subunits of other self-assembling systems of biological molecules is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686; 5,910,403; and 6,010,846 which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to those of skill in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley

The comparison of excess molar ratios to the theoretical patterns can be carried out using a table generated for a protein or phospholipid or galactocerebroside or cholesterol of interest, or graphically, using determined relationships. From these comparisons, a value, such as the value p, is determined, which describes the probability of mass isotopic enrichment of a subunit in a precursor subunit pool. This enrichment is then used to determine a value, such as the value Aₓ*, which describes the enrichment of newly synthesized proteins or phospholipids or galactocerebroside or cholesterol for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer Mₓ,

Fractional abundance of

$${M}_{X} = {A}_{X} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

$$Δ Fractional abundance ( enrichment or depletion ) = {( {A}_{x} )}_{e} - {( {A}_{x} )}_{b} = {( \frac{Abundance {M}_{x}}{{\sum }_{i = 0}^{n} Abundance {M}_{i}} )}_{e} - {( \frac{Abundance {M}_{x}}{{\sum }_{i = 0}^{n} Abundance {M}_{i}} )}_{b} ,$$

where subscript e refers to enriched and b refers to baseline or natural abundance.

In order to determine the fraction of molecular assemblages that were actually newly formed during a period of precursor administration, the measured excess molar ratio (EMₓ) is compared to the calculated enrichment value, Aₓ*, which describes the enrichment of newly formed molecular assemblage for each mass isotopomer, to reveal the isotopomer excess ratio which would be expected to be present, if all isotopomers were newly formed.

### Calculating Molecular Flux Rates

The method of determining the rate of self-assembly includes calculating the proportion of mass isotopically-labeled subunit of a molecular assemblage present in the precursor pool, and using this proportion to calculate an expected frequency of a molecular assemblage containing at least one mass isotopically-labeled subunit of a molecular assemblage. This expected frequency is then compared to the actual, experimentally determined isotopomer frequency. From these values, the proportion of molecular assemblage which is self-assembled from added isotopically-labeled precursors during a selected incorporation period can be determined. Thus, the rate of self-assembly during such a time period is also determined. In a system at steady-state concentrations, or when any change in concentrations in the system are measurable or otherwise known during said time period, the rate of disassembly is thereby known as well, using calculations known in the art. A precursor-product relationship is then applied. For the continuous labeling method, the isotopic enrichment is compared to asymptotic (e.g., maximal possible) enrichment and kinetic parameters (e.g., synthesis rates) are calculated from precursor-product equations. The fractional synthesis rate (kₛ) may be determined by applying the continuous labeling, precursor-product formula:

kₛ=[−In(1−f)]/t,
where f=fractional synthesis=product enrichment/asymptotic precursor/enrichment
and t=time of label administration of contacting in the system studied.

For the discontinuous labeling method, the rate of decline in isotope enrichment is calculated and the kinetic parameters of subunits are calculated from exponential decay equations. In practicing the method, molecular assemblages are enriched in mass isotopomers, preferably containing multiple mass isotopically labeled subunits of self-assembling systems of biological molecules. These higher mass isotopomers of the molecular assemblage (e.g., proteins containing 3 or 4 mass isotopically labeled tubulin dimers for microtubule polymers) are formed in negligible amounts in the absence of exogenous precursor (e.g., ²H₂O), due to the relatively low abundance of natural mass isotopically-labeled precursor (e.g., ²H₂O), but are formed in significant amounts during the period of precursor incorporation (e.g., during administration of ²H₂O to the cell, tissue, organ, or organism). The molecular assemblages taken from the cell, tissue, organ, or organism at the sequential time points are analyzed by mass spectrometry, to determine the relative frequencies of a high mass isotopomer or to determine the relative frequencies of a high mass isotopomer of a subunit (e.g., protein, phospholipids, galactocerebroside, cholesterol) from a molecular assemblage. Since the high mass isotopomer is synthesized almost exclusively before the first time point, its decay between the two time points provides a direct measure of the rate of decay of the subunit. The rate of decay of mass isotopomers that do not contain multiple mass isotopically labeled subunits can also be calculated and used by the methods described herein.

Preferably, the first time point is at least 2-3 hours after administration of precursor (e.g., ²H₂O) has ceased, depending on mode of administration, to ensure that the proportion of mass isotopically labeled subunit (e.g., a labeled tubulin dimer for a microtubule polymer) has decayed substantially from its highest level following precursor administration. In one embodiment, the following time points are typically 1-4 hours after the first time point, but this timing will depend upon the replacement rate of the biopolymer pool.

The rate of decay of the molecular assemblage is determined from the decay curve for the isotope-labeled subunit. In the present case, where the decay curve is defined by several time points, the decay kinetics can be determined by fitting the curve to an exponential decay curve, and from this, determining a decay constant.

Breakdown rate constants (k_{d}) may be calculated based on an exponential or other kinetic decay curve:

k_{d}=[−In f]/t.

### USES OF THE PRESENT INVENTION

The invention finds use as an in vivo method of high-throughput screening for inhibitors of self-assembly or disassembly of self-assembling systems of biological molecules, and other higher-order structures (e.g., identifying and characterizing new microtubule-targeted tubulin-polymerizing agents).

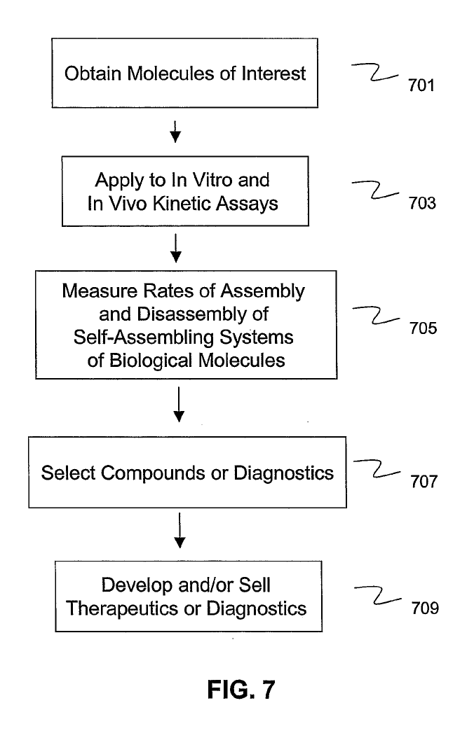

For example, a compound that inhibits disassembly of microtubules leads to the arrest of the cell cycle at metaphase thereby blocking mitosis and inhibiting cell replication and division (proliferation). Such a result is useful in cancer because tumor cells are characterized by their uncontrolled proliferation. Current microtubule-targeted tubulin-polymerizing agents (MTPAs) are important cancer chemotherapeutic agents that are effective in the treatment of many types of cancers, including carcinoma of the ovary, lung, head and neck, bladder, and esophagus. However, all known MTPAs have considerable deficiencies and identifying and characterizing new MTPAs with a more favorable therapeutic index would significantly advance the field of cancer chemotherapeutics. The methods of the present invention allow for the screening of compounds for activity on microtubule dynamics and therefore allow for the identification, evaluation, characterization, development, and marketing of compounds that treat these proliferative diseases such as cancer or psoriasis (FIG. 7 provides a roadmap of this process).

In addition, microtubule formation and disassembly play a key component in flagellar and ciliar movement of eucaryotic, procaryotic and archebacteria. Thus, for example, microtubule formation in sperm cells can be analyzed for increasing motility (e.g., fertility) or decreasing motility (e.g., birth control). Ciliated epithelial and endothelial cells play crucial roles in the respiratory and gastrointestinal tracts, and these cells can be evaluated for both toxicity and drug candidate action. Additionally, many, if not most, bacteria have flagella or cilia associated with movement, and testing compounds for antibiotic activity is also within the scope of the present invention.

Compounds that inhibit actin polymerization (e.g., microfilament) also may be useful in treating cancer, as actin dynamics are involved in cell motility/migration (e.g., metastasis of tumor cells) and adhesion of tumor cells. The methods of the present invention allow for the screening, identification, characterization, evaluation, development, and marketing of compounds to treat cancer and other disorders implicated by changes in the stability of microfilaments (e.g., muscular dystrophies, cardiomyopathies—see below). Additionally, the reorganization of the actin cytoskeleton (e.g., microfilament) is essential for angiogenesis. Any compound that affects microfilament dynamics will have an effect on endothelial cell proliferation and migration (e.g., angiogenesis). Specifically, microfilament dynamics are essential for movement-generating lamellipodia and membrane ruffles in response to one or more growth factors (which can be measured and compared with cells not exposed to one or more growth factors). Furthermore, since microfilament dynamics are also involved in several other diseases including various muscular dystrophies, various cardiomyopathies, myasthenia gravis, and amyotrophic lateral sclerosis, the methods of the present invention allow for the screening of compounds for activity on microfilament dynamics and therefore allow for the identification, evaluation, characterization, development, and marketing of compounds to treat these diseases (FIG. 7 provides a roadmap of this process).

Compounds that inhibit A□ self-assembly into A□ fibrils or plaques may be useful in treating Alzheimer's disease. The methods of the present invention allow for the screening, identification, characterization, evaluation, development, and marketing of compounds to treat Alzheimer's disease and other neurodegenerative disorders (e.g., dementia) implicated by A□ fibril or plaque formation (FIG. 7 provides a roadmap of this process).

Compounds that inhibit □-synuclein self-assembly into Lewy body filaments (Lewy bodies) and Lewy neurite filaments (Lewy neuritis) may be useful in treating Parkinson's disease and dementia (see B S, Jakes R, Tsutsui M, Spillantini M G, Crowther R A, Goedert M, Koto A. Brain Pathol. 2004 April; 14(2):137-47; Spillantini M G, Crowther R A, Jakes R, Hasegawa M, Goedert M. Proc Natl Acad Sci USA. 1998 May 26; 95(11):6469-73, both herein incorporated by reference). The methods of the present invention allow for the screening, identification, characterization, evaluation, development, and marketing of compounds to treat Parkinson's disease and other neurodegenerative disorders such as dementia implicated by the presence of Lewy bodies and Lewy neurites (FIG. 7 provides a roadmap of this process).

Compounds that inhibit tau self-assembly into tau filaments (plaques and neurofibrillary tangles) may be useful in treating Alzheimer's disease and dementia (for reviews see Chen F, David D, Ferrari A, Gotz J. Curr Drug Targets. 2004 August; 5(6):503-15; Goedert M, Spillantini M G, Serpell L C, Berriman J, Smith M J, Jakes R, Crowther R A. Philos Trans R Soc Lond B Biol Sci. 2001 Feb. 28; 356(1406):213-27, both herein incorporated by reference). The methods of the present invention allow for the screening, identification, characterization, evaluation, development, and marketing of compounds to treat Alzheimer's disease and other neurodegenerative disorders such as dementia implicated by the presence of tau filaments—diseases associated with tau molecular assemblages are collectively termed “tauopathies”—(FIG. 7 provides a roadmap of this process).

Compounds that inhibit prion self-assembly into prion fibrils or plaques may be useful in treating Creutzfeldt-Jakob disease, kuru, scrapies, or bovine spongiform encephalopathy. The methods of the present invention allow for the screening of compounds for activity on prion aggregation and therefore allow for the identification, evaluation, characterization, development, and marketing of compounds to treat prion diseases such as Creutzfeldt-Jakob disease, kuru, scrapies, or bovine spongiform encephalopathy (FIG. 7 provides a roadmap of this process).

Compounds that inhibit fibrin self-assembly into fibrin aggregates (clots or thrombi) may be useful in treating diseases or conditions involving excess or insufficient thrombi formation such as pulmonary embolism, deep-venous thrombosis, stroke and myocardial infarction (excess thrombi formation) or bleeding disorders such as hemophilia, disseminated intravascular coagulation, vitamin K deficiency or liver disease (insufficient thrombi formation). The methods of the present invention allow for the screening of compounds for activity on fibrin aggregate formation and therefore allow for the identification, evaluation, characterization, development, and marketing of compounds to treat myocardial infarction, stroke, bleeding disorders and other diseases or conditions involving alterations in thrombus formation (FIG. 7 provides a roadmap of this process).

Compounds that inhibit mutant hemoglobin self-assembly into mutant hemoglobin aggregates in erythrocytes causing the characteristic sickling in sickle-cell anemia may be useful in treating sickle-cell anemia and other diseases characterized at least partially by mutant hemoglobin aggregation. The methods of the present invention allow for the screening of compounds for activity on mutant hemoglobin aggregation and therefore allow for the identification, evaluation, characterization, development, and marketing of compounds to treat sickle-cell anemia and other diseases at least partially characterized by aggregates of mutant hemoglobin (FIG. 7 provides a roadmap of this process).

Compounds that increase the amount of microtubule polymers and promote net incorporation of newly synthesized tubulin into microtubules in neurons produce neurotoxic effects including neuropathies. The methods of the present invention allow for the screening of compounds for neurotoxic effects including neuropathies. Using the methods of the present invention, the skilled artisan could test a group of compounds (for example, all congeners of a drug in development) and select the least neurotoxic congener for further development as the preferred agent (FIG. 7 provides a roadmap of this process).

Compounds that stabilize the aggregation of certain phospholipids (e.g., phosphatidylcholine) in myelin lamellae may find use in the treatment of multiple sclerosis and other demyelinating diseases since it has been shown that a decrease in phosphatidylcholine content in myelin lamellae contributes to demyelination (see, e.g., Ohler B, Graf K, Bragg R, Lemons T, Coe R, Genain, Israelachvili J, Husted C. Biochim Biophys Acta. 2004 Jan. 20; 1688(1):10-7, herein incorporated by reference). Compounds that stabilize galactocerebroside aggregation in myelin lamellae will serve the same purpose as compounds that stabilize the aggregation of phosphatidylcholine. Alternatively, compounds that decrease stability or increase instability of aggregates of certain other phospholipids (such as phosphatidylserine) in myelin lamellae also may find use in treating multiple sclerosis, as increased phosphatidylserine content in myelin lamellae has been shown to promote demyelination (see, e.g., Ohler B, Graf K, Bragg R, Lemons T, Coe R, Genain C, Israelachvili J, Husted C. Biochim Biophys Acta. 2004 January 20; 1688(1):10-7, previously incorporated by reference) (FIG. 7 provides a roadmap of this process).

Disruptions of the plasma membrane lead to muscular dystrophies. Although one approach to treating the muscular dystrophies has been discussed, supra, (e.g., stabilizing the actin microfilaments comprising the cytoskeleton), compounds that stabilize the plasma membrane by stabilizing aggregates of phospholipids and/or cholesterol may also be useful in treating the muscular dystrophies. The methods of the present invention allow for the identification, characterization, evaluation, development, and marketing of such compounds that stabilize phospholipids and/or cholesterol in the plasma membrane, which may find use in treating muscular dystrophies (FIG. 7 provides a roadmap of this process).

The methods of the present invention also allow for the measurement of the lifespan of the lipid bilayer of the plasma membrane or any of the lipid membranes surrounding the subcellular organelles. Labeled cholesterol or labeled phospholipid incorporated into the membrane can be compared to free labeled cholesterol or free labeled phospholipid or both comparisons may be conducted simultaneously (e.g., cholesterol and phospholipid) to determine the lifespan of the lipid membrane (the molecular assemblage of interest). Compounds can then be screened to determine whether they are stabilizing or destabilizing to a lipid membrane of interest (e.g., plasma membrane, endoplasmic reticulum membrane, golgi apparatus membrane, lysozyme membrane, lysosome membrane, any vesicle membrane such as a neurotransmitter vesicle membrane or a secretory protein vesicle membrane, nuclear membrane, outer mitochondrial membrane, and inner mitochondrial membrane)(FIG. 7 provides a roadmap of this process).

The methods of the present invention also allow for the measurement of the lifespan of the inner mitochondrial membrane (the molecular assemblage of interest). By using the methods of the present invention, labeled cardiolipin incorporated in the inner membrane of the mitochondrion can be compared with free labeled cardiolipin. Compounds can then be screened to determine whether they are stabilizing or destabilizing to the inner membrane of the mitochondrion (FIG. 7 provides a roadmap of this process).

Although the foregoing has provided illustrative examples of uses of the present invention, such examples of uses are not limiting as one of skill in the art would recognize that the methods of the present invention can be applied to other examples of uses of the present invention for identifying, evaluating, characterizing, developing, and marketing compounds for treating diseases associated with self-assembling systems of biological molecules.

FIG. 7 illustrates the uses of the invention herein in a drug discovery and development process. At step 701 a plurality of drug candidates or other compounds are obtained, for example by purchase or in-licensing. At step 703 the compounds are applied to the in vitro and in vivo kinetic assays as described herein. At step 705 the dynamics of assembly and disassembly of self-assembling systems of biological molecules are measured as described herein. If it is desirable to reduce the dynamics in a particular phenotypic state, a compound that reduces the dynamics of assembly and disassembly, for example by stabilizing the assembled molecular assemblage or inhibiting subunit incorporation into the molecular assemblage will be considered generally more useful, and conversely a compound that increases the dynamics will be considered generally less desirable. In a target discovery process, a particular phenotype that has increased or decreased dynamics with respect to another phenotype (e.g., diseased vs. not diseased) may be considered a good therapeutic or diagnostic target or in the pathway of a good therapeutic or diagnostic target. At step 707 compounds of interest, targets of interest, or diagnostics are selected and further used and further developed. In the case of targets, such targets may be the subject of, for example, well known small molecule screening processes (e.g., high-throughput screening of new chemical entities) and the like. Alternatively, biological factors, or already-approved drugs, or other compounds (or combinations and/or mixtures of compounds) may be used. At step 709 the compounds or diagnostics are sold or distributed. It is recognized of course that one or more of the steps in the process in FIG. 7 will be repeated many times in most cases for optimal results.

### Isotopically-Perturbed Molecules or Molecular Assemblages

In another variation, the methods provide for the production of isotopically-perturbed molecules (e.g., labeled amino acids, labeled peptides, labeled proteins, labeled phospholipids, labeled cholesterol, labeled cardiolipin and the like) or molecular assemblages (e.g., microtubules containing isotopically perturbed tubulin dimers; amyloid fibril aggregates containing isotopically perturbed A□ peptides). These isotopically-perturbed molecules comprise information useful in determining the flux of molecules within the assembly/disassembly pathway of the molecular assemblage. Once isolated from a cell and/or a tissue of an organism, one or more isotopically-perturbed molecules are analyzed to extract information as described, supra.

### Kits

The invention provides kits for measuring and comparing molecular flux rates in vivo. The kits may include isotope-labeled precursor molecules, and in preferred embodiments, chemical compounds known in the art for separating, purifying, or isolating proteins, and/or chemicals necessary to obtain a tissue sample, automated calculation software for combinatorial analysis, and instructions for use of the kit.

Other kit components, such as tools for administration of water (e.g., measuring cup, needles, syringes, pipettes, IV tubing), may optionally be provided in the kit. Similarly, instruments for obtaining samples from the living system (e.g., specimen cups, needles, syringes, and tissue sampling devices) may also be optionally provided.

### Information Storage Devices

The invention also provides for information storage devices such as paper reports or data storage devices comprising data collected from the methods of the present invention. An information storage device includes, but is not limited to, written reports on paper or similar tangible medium, written reports on plastic transparency sheets or microfiche, and data stored on optical or magnetic media (e.g., compact discs, digital video discs, optical discs, magnetic discs, and the like), or computers storing the information whether temporarily or permanently. The data may be at least partially contained within a computer and may be in the form of an electronic mail message or attached to an electronic mail message as a separate electronic file. The data within the information storage devices may be “raw” (e.g., collected but unanalyzed), partially analyzed, or completely analyzed. Data analysis may be by way of computer or some other automated device or may be done manually. The information storage device may be used to download the data onto a separate data storage system (e.g., computer, hand-held computer, and the like) for further analysis or for display or both. Alternatively, the data within the information storage device may be printed onto paper, plastic transparency sheets, or other similar tangible medium for further analysis or for display or both.

### Robotic Components

In a preferred embodiment, for example when cell culture systems, including bacterial culture systems are used, the devices of the invention can comprise liquid handling components, including components for loading and unloading fluids at each station or sets of stations. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; holders with cartridges and/or caps; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention, for example for purification of assemblages or subunits.

In a preferred embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of heat exchangers such as controlled blocks or platforms to provide accurate temperature control of incubating samples from 0° C. to 100° C.; this is in addition to or in place of the station thermocontrollers.

In a preferred embodiment, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

### EXAMPLES

The following non-limiting examples further illustrate the invention disclosed herein:

### Example 1

### Stable Isotope Incorporation into Tubulin Dimers and Microtubule Polymers Reveals Paclitaxel Effects on Microtubule Dynamics in Cultured Human Lung Cancer Cells (SW1573)

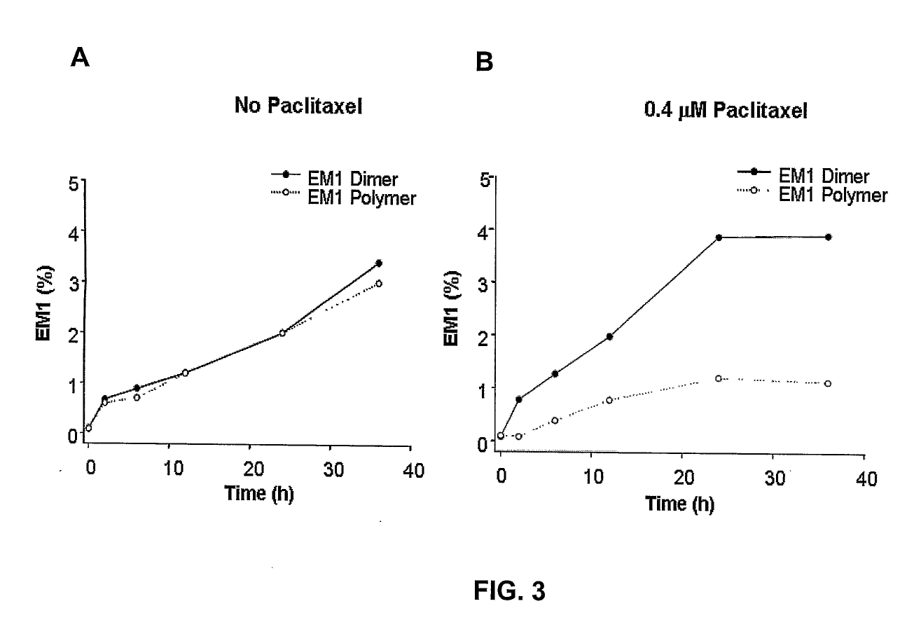

To determine whether biosynthetic labeling with ²H₂O can reveal MTPA effects on microtubule dynamics in actively proliferating cells, SW1573 human lung cancer cells were cultured and labeled during exponential growth with culture media containing 4% ²H₂O for 2, 6, 12, 24 and 36 hr, respectively. Tubulin dimer and polymer fractions were isolated from post-nuclear supernatants, and tubulin was purified from each fraction to ≧90% purity as determine by SDS-PAGE. After acid hydrolysis, amino acids were derivatized for GC/MS analysis with negative chemical ionization, and ²H-label incorporation into the alanine derivative was quantified as the increase over natural abundance of the (M+1) mass isotopomer (“²H—enrichment”; FIG. 3). Label incorporation steadily increased during 36 hours time (FIG. 3A), indicating ²H—alanine incorporation into newly synthesized tubulin dimer and polymer fractions. The similar rates of ²H—tubulin incorporation in both fractions indicated that the flux of dimer into polymer occurred in a time scale that was fast compared to the overall rate of label incorporation. The data support that in actively dividing cells, tubulin dimers and polymers are in a kinetic equilibrium on a time scale of hours. In contrast, in the presence of 0.4 □M paclitaxel, the rate of ²H incorporation into polymers was reduced by 75% compared to the rate of label incorporation into the dimer (FIG. 3B). This indicates a substantial inhibition of ²H—tubulin dimer flux into polymer, reflective of the ability of paclitaxel to stabilize microtubules and thus to prevent dynamic instability.

### Example 2

### Stable Isotope Incorporation Reveals Effects of Paclitaxel on Microtubule Dynamics in vivo

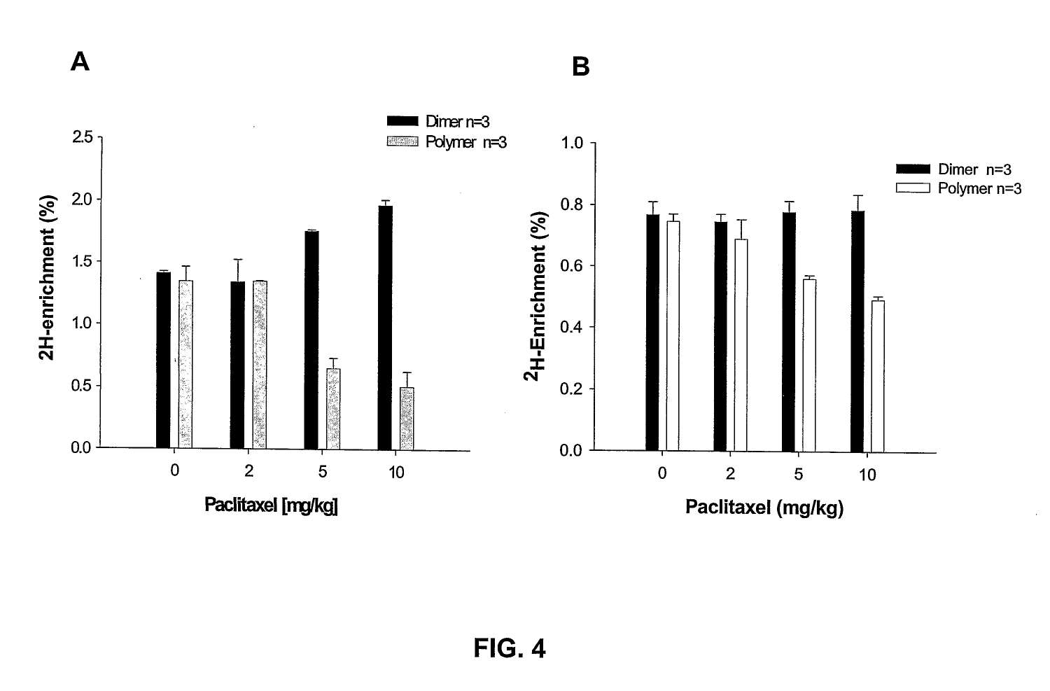

To demonstrate the feasibility of applying the methods of the present invention of measuring microtubule dynamics in vivo, SW1573 human lung cancer cells and MCF-7 human breast cancer cells were implanted separately into nude mice. The tumors were allowed to grow to approximately 1000 mm³ in diameter; mice were then injected intraperitoneally with increasing doses of paclitaxel. ²H₂O (8%) in drinking water was administered for a 24 hr period, resulting in about 5% ²H enrichment in body water (the balance being metabolic water). Animals were sacrificed 24 hours after drug treatment, and tumor tissue was removed for analysis of ²H—label incorporation into tubulin dimers and polymers (FIG. 4). In SW1573 tumors from control animals, relative synthesis was reduced compared to what was observed in vitro culture (1.4% ²H—enrichment, FIG. 4A, versus 2% at 24 hr in FIG. 3), but importantly, label incorporation into tubulin dimer and microtubules was again indistinguishable, reflecting rapid exchange between the two pools (highly dynamic microtubules). In contrast, paclitaxel treatment decreased incorporation of ²H—tubulin into microtubules in a dose-dependent manner indicating suppressed dynamicity, whereas fractional synthesis of tubulin dimers increased, similarly to what was measured in culture (FIG. 3B).

Qualitatively similar effects of paclitaxel were also seen in implanted MCF-7 tumors, though the amount of ²H—label incorporation was lower than in SW1573 tumors (FIG. 4B). Paclitaxel-dependent reduction of ²H—label polymer was less pronounced, albeit statistically significant at the 5 and 10 mg/kg doses. Moreover, there was no significant paclitaxel-induced upregulation of tubulin fractional synthesis over baseline in this tumor.

The data support the finding that the stable isotope labeling assay is capable of quantifying the modulation of microtubule dynamics by paclitaxel in vivo and that paclitaxel effects in vivo are similar to those observed in cell culture.

### Example 3

### Comparing Microtubule Dynamics to Cell Proliferation

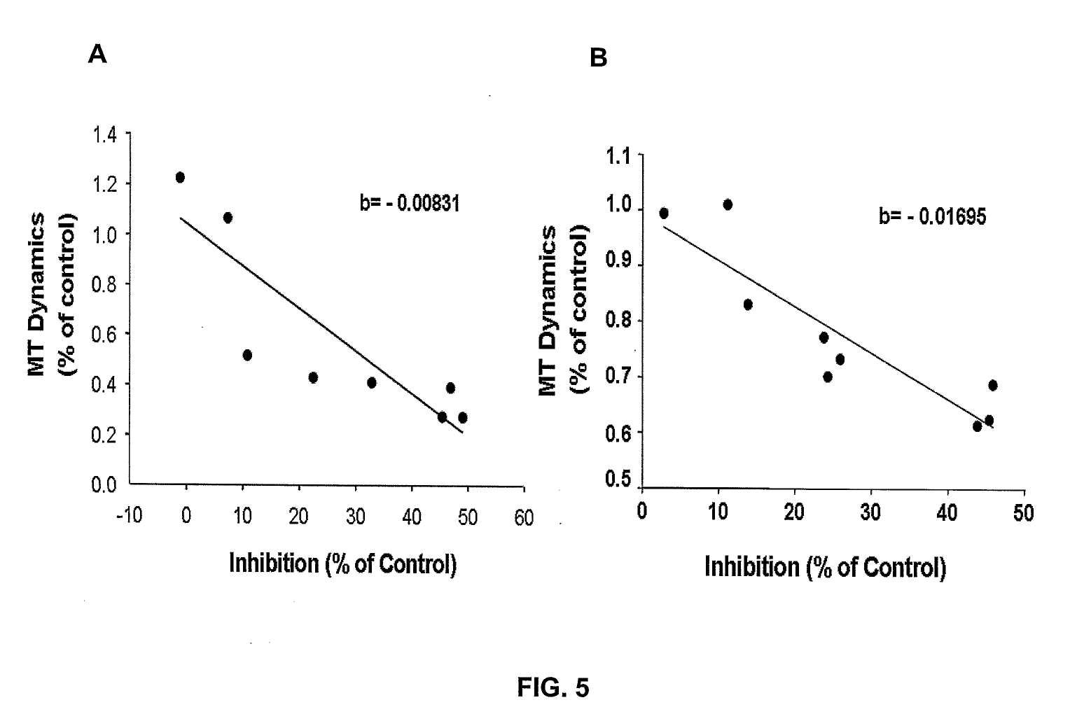

Various factors can interfere with the actions of paclitaxel in tumors, including alterations in tubulin isotype content, efflux pumps, and in vivo drug metabolism. Regardless of these factors, inhibition of microtubule dynamics, if related to the antiproliferative activity of MTPAs in vivo, should correlate with inhibition of cell proliferation. To explore this relationship, paclitaxel-induced inhibition of label incorporation into microtubules was correlated with the inhibition of fractional DNA synthesis in tumor cell tissue (the latter serving as a convenient stable isotope-based measure of cell proliferation, (see U.S. Pat. Nos. 5,910,403, 6,010,846, 6,461,806, previously incorporated by reference). The results (FIGS. 5A and 5B) show a strong relationship, in both types of tumor, between inhibition of microtubule dynamics (expressed as fractional loss, compare to controls, of label incorporation into polymerized microtubules) and inhibition of newly synthesized DNA (a measure of tumor cell proliferation). Thus, the inhibition of microtubule dynamics appeared to have a measurable impact on and relation to the in vivo antiproliferative action of MTPAs.

### Example 4

### Paclitaxel-Induced Cytotoxicity and NNeuropathy

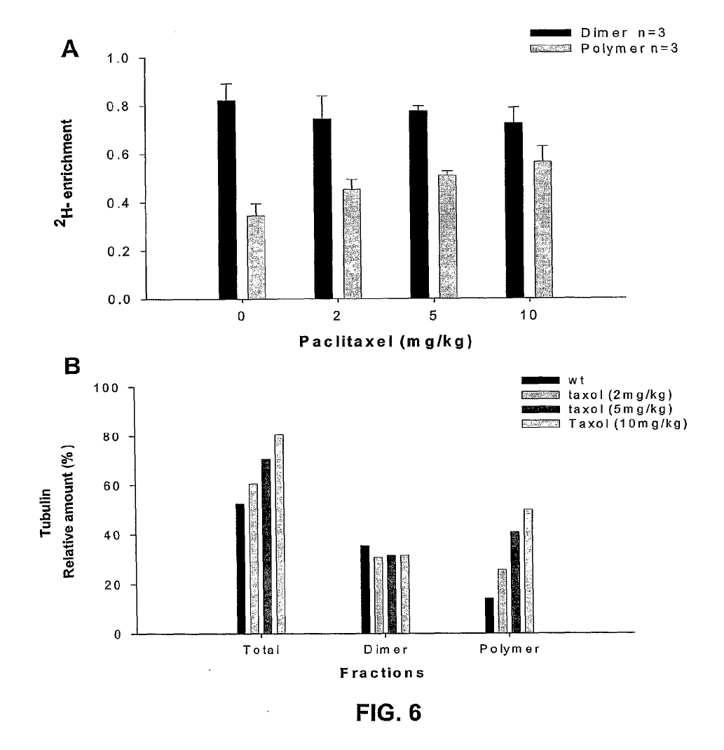

In order to evaluate neurotoxic effects of paclitaxel, the sciatic nerve was isolated from tumor-bearing mice and ²H label incorporation into free tubulin was compared with ²H label incorporation into polymerized microtubuies (FIG. 6). The results contrasted remarkably with those observed in tumor cells. Unlike the kinetic equilibrium between free and bound tubulin observed in tumor cells, sciatic nerve microtubules were largely static at baseline, and ²H label incorporation into polymer was only about 40% of that measured in free tubulin (FIG. 6A). Surprisingly, higher doses of paclitaxel increased the label incorporation into polymers, while slightly decreasing labeling of free tubulin, indicating that a greater fraction of newly made tubulin ends up in microtubules after drug administration. To explore this effect further, net changes in the abundance of unpolymerized free tubulin and microtubules was assessed by densitometric analysis (FIG. 6B). The data revealed a dose-dependent increase in the amount of microtubule polymers and a slight decrease in free tubulin dimers indicating that, in sciatic nerves, paclitaxel increased the amount of microtubule polymers and promoted net incorporation of newly synthesized tubulin into microtubules.

### Example 5

### Screening Compounds for Ability to Inhibit Microtubule Dynamics

Determining whether a new chemical entity (NCE), or combinations of NCEs, drug candidate, or combinations of drug candidates, drug lead, or combinations of drug leads, or an already-approved drug such as one listed in the Physician's Desk Reference (PDR) or Merck Index, or combinations of already-approved drugs, or a biological factor, or combinations of biological factors (or any combination of mixtures of NCEs, drug candidates, drug leads, already-approved drugs, and/or biological factors) can inhibit microtubule dynamics is important in determining whether an NCE, or a drug candidate, or a drug lead, or an already-approved drug, or a biological factor (or combinations of NCEs, or combinations of drug candidates, or combinations of drug leads, or combinations of already-approved drugs, or combinations of biological factors, or combinations of various mixtures of NCEs and/or drug candidates and/or drug leads and/or already-approved drugs and/or biological factors) has potential for treating cancer.

To assess whether an NCE, or a drug candidate, or a drug lead, or an already-approved drug, or a biological factor (or combinations encompassing NCEs, drug candidates, drug leads, already-approved drugs, and biological factors, or combinations encompassing any variation thereof including any mixtures thereof) inhibits microtubule dynamics (and therefore, as stated above, a candidate drug specific for treating cancer) SW1573 human lung cancer cells and MCF-7 human breast cancer cells are implanted separately into nude mice. The tumors are allowed to grow to approximately 1000 mm³ in diameter; mice are then injected intraperitoneally with increasing doses of compound or a combination of compounds. ²H₂O (8%) in drinking water is administered for a 24 hr period, which results in about 5% ²H enrichment in body water (the balance being metabolic water). Animals are sacrificed 24 hours after treatment with compound, and tumor tissue is removed for analysis of ²H—label incorporation into tubulin dimers and polymers as described, supra. Further development of compounds showing activity is then undertaken as is depicted in FIG. 7. As indicated in FIG. 7, the methods of the invention are applicable to the discovery, development, and marketing of any compound having activity on any self-assembling system of biological polymers such as actin polymerization into actin microfilaments, A□ aggregation into A□ fibrils or plaques, fibrin assembly into blood clots, mutant hemoglobin aggregation in sickled erythrocytes, and prion aggregation into prion fibrils or plaques.

### Example 6

### Measuring Actin Self Assembly Using Stable-Isotope Labeling

The incorporation of a stable-isotope label substrate into one or more actin monomers (subunits of actin filaments) and the incorporation into actin filaments (e.g., microfilaments) are measured concurrently, such that the dynamics of actin assembly and disassembly (polymerization and depolymerization) can be calculated.

To isolate actin monomers and actin filaments several methods described in the literature are combined (Segura, M., and U. Lindberg. (1984): J. Biol. Chem. 259:3949-3954; Ohshima, S., H. Abe, and T. Obinata. (1989): J. Biol. Chem. 105:855-7; Pinder, J. C., J. A. Sleep, P. M. Bennett, and W. B. Gratzer. (1995): Anal. Biochem. 225:291-295, herein incorporated by reference in their entirety). This protocol differs from the conventional purification of muscle actin in that subjecting the actin to alternate cycles of polymerization and depolymerization is not required. Briefly, the preparation includes extraction and dissociation of actin complexes in cells with high concentrations of Tris buffer; multiple, high-speed centrifugations; and anion exchange chromatography and affinity chromatography on DNase I-Sepharose. Actin from adult chicken brain, bovine erythrocytes and chick embryo brain has successfully been purified by this method, which can be applied to a variety of tissues or cultured cells. The incorporation of stable-isotope label substrate into one or more actin monomers (subunits of actin filaments) and the incorporation into actin filaments (i.e. microfilaments) are measured, such that the dynamics of actin assembly and disassembly (polymerization and depolymerization) can be calculated.

### Example 7

### Measuring Tau Self-Assembly into Filaments using Stable-Isotope Labeling

The protein tau self-assembles into a polymer scaffold known as paired helical filaments (PHF or Alzheimer neurofibrillary tangles). The incorporation of stable-isotope label substrate into one or more tau protein (subunits of PHF) and the incorporation into paired helical filaments (i.e. PHF) are measured concurrently, such that we can calculate the rate of tau self-aggregation into PHF (tau polymerization). To isolate soluble tau proteins and PHF-tau, several methods are combined that are described in the literature (Grundke-Igbal I, Igbal K, Quinlan M, Tung Y C, Zaidi M S, Wisniewski H M. (1986):J Biol Chem. 261:6084-9; Wischik C M, Novak M, Thogersen H C, Edwards P C, Runswick M J, Jakes R, Walker J E, Milstein C, Roth M, Klug A (1988): Proc Natl Acad Sci USA. 85:4506-10, herein incorporated by reference in their entirety). The AD brain cytosolic extract low-speed supernatant is used to enrich soluble tau proteins and the pellet to enrich the tau polymers (PHF-tau). Cytosolic extract low-speed supernatant is heated up to 80-90° C, sonicated and incubated in a boiling water bath for 5 min, followed by high-speed centrifugation. Supernatant is sequentially treated with 2.5% perchloric acid and 6% trichloroacetic acid on ice for 30 min, followed by high-speed centrifugation to sediment pure soluble tau-enriched fraction (pellet). Cytosolic extract low-speed pellet (crude PHF) is sonicated at 100° C. in 10 volumes of 0.8M NaCl, and fractionated by multiple high-speed centrifugations in 0.16M and 0.5M sucrose cushions followed by 1% (wt/vol) sarkosyl cushion to sediment pure PHF-tau (tau polymers).

### Example 8

### Measuring Aβ Self-Assembly into Protofibrils and Fibrils using Stable-Isotope Labeling

Aβ₍₁₋₄₀₎ and/or Aβ₍₁₋₄₂₎ peptides (monomers) can self-assemble into protofibrils and fibrils (amyloid plaques) in a process called fibrillization or amyloidogenesis. The incorporation of stable-isotope label substrate into one or more Aβ₍₁₋₄₀₎ and/or Aβ₍₁₋₄₂₎ monomers (subunits of amyloid plaques) and the incorporation into protofibrils and subsequent fibrils (e.g., plaques) are measured concurrently, such that the rate of Aβ₍₁₋₄₀₎ and/or Aβ₍₁₋₄₂₎ monomers self-aggregation into amyloid plaques (fibrillization) can be calculated. To isolate soluble Aβ monomers/dimers or unstable intermediates, protofibrils (oligomers) and fibrils (plaques) several methods described in the literature are combined (Cai, X. D., Golde, T. E. & Younkin, S. (1993): Science 259, 514-516; Johnson-Wood, K., Lee, M., Motter, R., Hu, K., Gordon, G., Barbour, R., Khan, K., Gordon, M., Tan, H., Games, D., et al. (1997) Proc. Natl. Acad. Sci. USA 94, 1550-1555; 15; Melissa A. Moss, Michael R. Nichols, Dana Kim Reed, Jan H. Hoh, and Terrone L. Rosenberry Mol Pharmacol 64:1160-1168, 2003, herein incorporated by reference in their entirety). Briefly, 70% formic acid will be use to extract soluble and insoluble Aβ from AD brains. After neutralization of pH with equal volume of 2 M Tris (pH 6.8), the soluble extracts are fractionated into monomers/dimers and protofibrils (oligomers) by size exclusion chromatography at cold temperature and in the presence of 0.2% Triton X-100 to reduce Aβ stickiness to the column. Isolated protofibrils (oligomers) are sonicated and further fractionated into monomers/dimers by HPLC size exclusion chromatography. The insoluble Aβ fibrils (plaques) are denatured by dissolution in 8 M urea, pH 10 followed by sonication in a 40° C. water bath sonicator for 1 h, followed by filtration through an Anotop 25 Plus 20-nm filter (Whatman). The soluble disassembled fibrils are fractionated into momomers/dimers by HPLC size exclusion chromatography.

### Example 9

### Measuring Tubulin Self-Assembly into Sperm Axonemal Microtubules using Stable-Isotope Labeling

Tubulin is present in all eukaryotes where it constitutes the building block of all classes of microtubules: the interphasic network of microtubules structuring all cytoplasmic transports of organelles, mitotic spindles, the 9 outer doublets and 2 central singlets of axonemes and the basal bodies and centriolar triplets (Dustin P. (1984): Microtubules. Springer-Verlag, Berlin 2nd ed; Alberts B., Bray D., Lewis J., Raff M., Roberts K., Watson J D. (1994): Molecular Biology of the Cell. Garland Publishing, New-York & London, 3rd Ed, herein incorporated by reference in their entirety).

Microtubules represent the main structural feature of the axoneme, anchored at its base on the distal centriole of the sperm basal body. Microtubule doublets present in axonemes are made according to the same rules by elongation of the distal centriole present in basal bodies by addition of tubulin dimers at the distal (plus) end into the A fiber (13 protofilaments) and the B fiber (11 protofilaments). The incorporation of stable- isotope label substrate into one or more tubulin dimers (subunits of microtubules) and the incorporation into microtubules (e.g., tubulin polymers) are measured concurrently, such that the rate of tubulin self-aggregation into microtubules (tubulin polymerization) can be calculated. Axonemal microtubules assembly can be inhibited by various microtubule-targeted tubulin-polymerizing (MTPAs) and depolymerizing (MTPDAs) agents. The method of the invention disclosed herein can be applied to screen for inhibitors and enhancers of sperm motility in vitro and in vivo.

To isolate sperm soluble tubulin dimers and axonemal microtubules several methods described in the literature are combined (Simon, J. R., N. A. Adam and E. D. Salmon (199): Micron Microsc. Acta. 22:405-412; Waterman-Storer, C. M. and E. D. Salmon (1997): Journal of Cell Biology. 139:417-434; Salmon, E. D., and Way, M. (1999): Cytoskeleton. Current Opinion in Cell Biology 11: 15-17, herein incorporated by reference in their entirety). Experimental animal or human sperm is collected by low-speed centrifugation (3,000×g) at 4° C. for 5 min. The sperm (pellet) is demembranated in 5 volumes of 20% sucrose in microtubule stabilizing buffer and gently homogenized using a Dounce glass homogenizer partially immersed in slushy ice. Sperm heads are separated form tails using alternate low-speed and high-speed ultracentrifugation steps (12,000×g for 10 min at 4° C. and 20,000×g for 15 min at 4° C.). Sperm tails (pellet) are stratified into a top white layer that contains the demembranated tails and a bottom yellow layer that contains heads and debris. The top white layer is collected and resuspended 4 volumes of microtubule stabilizing buffer. The cycle of resuspension and centrifugation is repeated 3 times to completely separate the tail fragments from the head and debris. The pure tail fragments (white pellet) are suspended in 4 volumes of microtubule stabilizing extraction buffer and transfer to a Dounce glass homogenizer on ice. The extract is incubated on ice for 45 min then fractionate by high-speed ultracentrifugation into soluble tubulin dimers (supernatant) and axonemal microtubules (pellet).

What is claimed is:

1. A method for measuring the rate of self assembly of subunits into biological molecular assemblages in a test living system as compared to a control living system, said method comprising:
a) administering an isotope-labeled substrate to said living system for a first period of time sufficient for said substrate to be incorporated into at least one of said subunits and at least one of said molecular assemblages;
b) obtaining a first sample from said living system;
c) quantifying the amount of labeled molecular assemblages from said first sample;
d) quantifying the amount of unincorporated labeled subunits;
e) comparing the amount of labeled molecular assemblages to the amount of labeled molecular assemblage in a control living system; and
f) comparing the amount of unincorporated labeled subunits to the amount of unincorporated labeled subunits in a control living system; to determine a difference in said rate of self assembly in said test living system as compared to said control living system.

2. The method according to claim 1 further comprising calculating the molecular flux rate of said labeled molecular assemblages, wherein said comparing step comprises calculating the ratio of said rates and comparing said ratio to the ratio of molecular flux rates in said control living system.

3. The method according to claim 1 wherein said isotope-labeled substrates are tubulin protein dimers.

4. The method of claim 1, wherein said isotope-labeled subunits are monomeric actin proteins.

5. The method of claim 1, wherein said isotope-labeled subunits are prion proteins.

6. The method of claim 1, wherein said isotope-labeled subunits are amyloid-beta proteins.

7. The method of claim 1, wherein said isotope-labeled subunits are fibrin proteins.

8. The method of claim 1, wherein said isotope-labeled subunits are mutant hemoglobin proteins.

9. A method according to claim 1 further comprising administering a candidate agent to said test living system.

10. A method according to claim 9 wherein said candidate agent is administered prior to said administration of said isotope-labeled substrate.

11. A method according to claim 9 wherein said candidate agent is administered during said administration of said isotope-labeled substrate.

12. A method according to claim 9 wherein said candidate agent is administered after said administration of said isotope-labeled substrate.

13. A method according to claim 1 further comprising administering said substrate for a second period of time and repeating steps b)-f).

14. A method according to claim 1 further comprising obtaining a second sample and repeating steps c)-f).

15. A methods according to claim 5 wherein said candidate agent is a drug.

16. The method of claim 1, wherein said isotope-labeled substrate is labeled with a stable isotope.

17. The method of claim 1, wherein said isotope-labeled substrate is stable-isotope labeled water.

18. The method of claim 17, wherein said stable-isotope labeled water is labeled with 2H.

19. The method of claim 1, wherein said isotope-labeled substrate is labeled with a radioactive isotope.

20. The method of claim 19, wherein said isotope-labeled substrate is radioactive-isotope labeled water.

21. The method of claim 20, wherein said radioactive isotope-labeled water is labeled with 3H.

22. The method of claim 1, wherein said test living system and said control living system are mammals.

23. The method of claim 1, wherein said test living system and said control living system are human.

24. The method of claim 1, wherein said test living system and said control living system are cell lines.

25. The method of claim 1, wherein said test living system and said control living system are primary cells.

26. The method of claim 25, wherein said primary cells are sperm cells.

27. The method of claim 1, wherein said test living system and said control living system are bacterial cells.
